SEQUENCE LISTING (268 sequences)

SEQ ID NO: 1 - DNA, 25 bp, synthetic construct
  source 1..25: mol_type = other DNA
taacactctaagcataactaaaggt

SEQ ID NO: 2 - DNA, 22 bp, synthetic construct
  source 1..22: mol_type = other DNA
cacaccattccccattgtgatt

SEQ ID NO: 3 - DNA, 22 bp, synthetic construct
  source 1..22: mol_type = other DNA
tgaccctaatctcttcgttgca

SEQ ID NO: 4 - DNA, 24 bp, synthetic construct
  source 1..24: mol_type = other DNA
tgtaaccaaggactcgtagctttt

SEQ ID NO: 5 - DNA, 16 bp, synthetic construct
  source 1..16: mol_type = other DNA
tgaggcccttggtgga

SEQ ID NO: 6 - DNA, 18 bp, synthetic construct
  source 1..18: mol_type = other DNA
cgctcgtccttgggaact

SEQ ID NO: 7 - DNA, 20 bp, synthetic construct
  source 1..20: mol_type = other DNA
tgcccgcattagctcttaga

SEQ ID NO: 8 - DNA, 20 bp, synthetic construct
  source 1..20: mol_type = other DNA
gtgcccgaggtggaagtact

SEQ ID NO: 9 - DNA, 24 bp, synthetic construct
  source 1..24: mol_type = other DNA
caacaacagggaccagataatttt

SEQ ID NO: 10 - DNA, 25 bp, synthetic construct
  source 1..25: mol_type = other DNA
cgtaccttactgagatctggagaca

SEQ ID NO: 11 - DNA, 18 bp, synthetic construct
  source 1..18: mol_type = other DNA
tgtcttcactggtgggcc

SEQ ID NO: 12 - DNA, 21 bp, synthetic construct
  source 1..21: mol_type = other DNA
agcccattcataatctggaca

SEQ ID NO: 13 - DNA, 21 bp, synthetic construct
  source 1..21: mol_type = other DNA
cgctctttgtctctgcctcat

SEQ ID NO: 14 - DNA, 19 bp, synthetic construct
  source 1..19: mol_type = other DNA
ctgctgtcctggctgcttt

SEQ ID NO: 15 - DNA, 22 bp, synthetic construct
  source 1..22: mol_type = other DNA
cttcaggaacagaacgaacagg

SEQ ID NO: 16 - DNA, 23 bp, synthetic construct
  source 1..23: mol_type = other DNA
ggagatgactagtttggcattca

SEQ ID NO: 17 - DNA, 20 bp, synthetic construct
  source 1..20: mol_type = other DNA
ggtagctgactgtgtgaaga

SEQ ID NO: 18 - DNA, 20 bp, synthetic construct
  source 1..20: mol_type = other DNA
cagtgttggagcagctcaat

SEQ ID NO: 19 - DNA, 17 bp, synthetic construct
  source 1..17: mol_type = other DNA
agcaggcccaagaggac

SEQ ID NO: 20 - DNA, 16 bp, synthetic construct
  source 1..16: mol_type = other DNA
ccgctgccaccgcttc

SEQ ID NO: 21 - DNA, 19 bp, synthetic construct
  source 1..19: mol_type = other DNA
ctgcatcgtcgctgtgtcc

SEQ ID NO: 22 - DNA, 24 bp, synthetic construct
  source 1..24: mol_type = other DNA
gttgtggtcattctctggaaatat

SEQ ID NO: 23 - DNA, 20 bp, synthetic construct
  source 1..20: mol_type = other DNA
gcacctcatttgccgaatac

SEQ ID NO: 24 - DNA, 20 bp, synthetic construct
  source 1..20: mol_type = other DNA
ctcctgcatcagcccaaata

SEQ ID NO: 25 - DNA, 17 bp, synthetic construct
  source 1..17: mol_type = other DNA
gtccgagtggctggagt

SEQ ID NO: 26 - DNA, 18 bp, synthetic construct
  source 1..18: mol_type = other DNA
accaccttctcgatggca

SEQ ID NO: 27 - DNA, 16 bp, synthetic construct
  source 1..16: mol_type = other DNA
tgcgagctccggcttt

SEQ ID NO: 28 - DNA, 22 bp, synthetic construct
  source 1..22: mol_type = other DNA
ccttgtagaaaccgtcagaatc

SEQ ID NO: 29 - DNA, 21 bp, synthetic construct
  source 1..21: mol_type = other DNA
accccactttccaatcacatc

SEQ ID NO: 30 - DNA, 20 bp, synthetic construct
  source 1..20: mol_type = other DNA
cgagtccgggtgtatctgaa

SEQ ID NO: 31 - DNA, 18 bp, synthetic construct
  source 1..18: mol_type = other DNA
tcctggcttcccgaaagt

SEQ ID NO: 32 - DNA, 22 bp, synthetic construct
  source 1..22: mol_type = other DNA
ggccaaagtcacagatcttcac

SEQ ID NO: 33 - DNA, 19 bp, synthetic construct
  source 1..19: mol_type = other DNA
atccgcgagagtgaaacca

SEQ ID NO: 34 - DNA, 21 bp, synthetic construct
  source 1..21: mol_type = other DNA
tcatcccaatcacggatagaa

SEQ ID NO: 35 - DNA, 23 bp, synthetic construct
  source 1..23: mol_type = other DNA
gcagcccgagtacttataattgg

SEQ ID NO: 36 - DNA, 22 bp, synthetic construct
  source 1..22: mol_type = other DNA
gatgagactgtgcaagtttcca

SEQ ID NO: 37 - DNA, 21 bp, synthetic construct
  source 1..21: mol_type = other DNA
tccgcatgactcataatttgc

SEQ ID NO: 38 - DNA, 21 bp, synthetic construct
  source 1..21: mol_type = other DNA
tgagggcgatagatttccatt

SEQ ID NO: 39 - DNA, 22 bp, synthetic construct
  source 1..22: mol_type = other DNA
ggaatggcatggccatcattag

SEQ ID NO: 40 - DNA, 21 bp, synthetic construct
  source 1..21: mol_type = other DNA
cgtggttgaacatgcaatagc

SEQ ID NO: 41 - DNA, 18 bp, synthetic construct
  source 1..18: mol_type = other DNA
cggcagcagaagatccgg

SEQ ID NO: 42 - DNA, 18 bp, synthetic construct
  source 1..18: mol_type = other DNA
cgctaggtgtcagcggct

SEQ ID NO: 43 - DNA, 20 bp, synthetic construct
  source 1..20: mol_type = other DNA
ttcttggcatcgcgaaagtg

SEQ ID NO: 44 - DNA, 25 bp, synthetic construct
  source 1..25: mol_type = other DNA
attttaaccacgttcttctccgata

SEQ ID NO: 45 - DNA, 18 bp, synthetic construct
  source 1..18: mol_type = other DNA
caaccaaggccgacaaaa

SEQ ID NO: 46 - DNA, 18 bp, synthetic construct
  source 1..18: mol_type = other DNA
tgatggcgggagtcacat

SEQ ID NO: 47 - DNA, 20 bp, synthetic construct
  source 1..20: mol_type = other DNA
tcctgctgacggaaattgtc

SEQ ID NO: 48 - DNA, 18 bp, synthetic construct
  source 1..18: mol_type = other DNA
acctccgggttggtcatc

SEQ ID NO: 49 - DNA, 19 bp, synthetic construct
  source 1..19: mol_type = other DNA
ccgagcgacgagagatcat

SEQ ID NO: 50 - DNA, 19 bp, synthetic construct
  source 1..19: mol_type = other DNA
caagggaccgcaccataag

SEQ ID NO: 51 - DNA, 20 bp, synthetic construct
  source 1..20: mol_type = other DNA
caacgaatggtactacggcc

SEQ ID NO: 52 - DNA, 21 bp, synthetic construct
  source 1..21: mol_type = other DNA
ggatgtatgatggccatgtca

SEQ ID NO: 53 - DNA, 18 bp, synthetic construct
  source 1..18: mol_type = other DNA
tgcccgaggaatggagtt

SEQ ID NO: 54 - DNA, 19 bp, synthetic construct
  source 1..19: mol_type = other DNA
cttgtgccaggaggacgtt

SEQ ID NO: 55 - DNA, 15 bp, synthetic construct
  source 1..15: mol_type = other DNA
gcgctggcgaaatcg

SEQ ID NO: 56 - DNA, 19 bp, synthetic construct
  source 1..19: mol_type = other DNA
ttcacgcgaaccagtgtca

SEQ ID NO: 57 - DNA, 21 bp, synthetic construct
  source 1..21: mol_type = other DNA
agtgaaacaagaggcggattc

SEQ ID NO: 58 - DNA, 21 bp, synthetic construct
  source 1..21: mol_type = other DNA
caagagacatccgggagaaaa

SEQ ID NO: 59 - DNA, 20 bp, synthetic construct
  source 1..20: mol_type = other DNA
gaatcaccgacatgctcaca

SEQ ID NO: 60 - DNA, 21 bp, synthetic construct
  source 1..21: mol_type = other DNA
ggatagcttggctcacgttct

SEQ ID NO: 61 - DNA, 20 bp, synthetic construct
  source 1..20: mol_type = other DNA
cagaagagccaggaatcgaa

SEQ ID NO: 62 - DNA, 21 bp, synthetic construct
  source 1..21: mol_type = other DNA
caactatgactccatggcgga

SEQ ID NO: 63 - DNA, 22 bp, synthetic construct
  source 1..22: mol_type = other DNA
tggagacccaagacatcaacat

SEQ ID NO: 64 - DNA, 17 bp, synthetic construct
  source 1..17: mol_type = other DNA
tcccccaacaatgctgc

SEQ ID NO: 65 - DNA, 19 bp, synthetic construct
  source 1..19: mol_type = other DNA
tgcacttctacggctggaa

SEQ ID NO: 66 - DNA, 20 bp, synthetic construct
  source 1..20: mol_type = other DNA
agccgctggtcttgtcctta

SEQ ID NO: 67 - DNA, 20 bp, synthetic construct
  source 1..20: mol_type = other DNA
tctggctttctttgcagcaa

SEQ ID NO: 68 - DNA, 21 bp, synthetic construct
  source 1..21: mol_type = other DNA
cttcttggctaaatcgctcca

SEQ ID NO: 69 - DNA, 19 bp, synthetic construct
  source 1..19: mol_type = other DNA
aggcacaggcttccttctg

SEQ ID NO: 70 - DNA, 21 bp, synthetic construct
  source 1..21: mol_type = other DNA
gcttgttccagtgagggagat

SEQ ID NO: 71 - DNA, 22 bp, synthetic construct
  source 1..22: mol_type = other DNA
ttcagaggagcccatttacatc

SEQ ID NO: 72 - DNA, 22 bp, synthetic construct
  source 1..22: mol_type = other DNA
ccttgagaaagtccagcaaact

SEQ ID NO: 73 - DNA, 22 bp, synthetic construct
  source 1..22: mol_type = other DNA
accagacctctacattccattt

SEQ ID NO: 74 - DNA, 24 bp, synthetic construct
  source 1..24: mol_type = other DNA
gataagaatttgtctcttcagtgt

SEQ ID NO: 75 - DNA, 20 bp, synthetic construct
  source 1..20: mol_type = other DNA
gctaaaagaactgacagccc

SEQ ID NO: 76 - DNA, 22 bp, synthetic construct
  source 1..22: mol_type = other DNA
aagaattgcaacagctcgattg

SEQ ID NO: 77 - DNA, 24 bp, synthetic construct
  source 1..24: mol_type = other DNA
cagtgaagaagacagcagcaaaaa

SEQ ID NO: 78 - DNA, 23 bp, synthetic construct
  source 1..23: mol_type = other DNA
agctggatcccttttagttcctt

SEQ ID NO: 79 - DNA, 21 bp, synthetic construct
  source 1..21: mol_type = other DNA
atcacatcgagccactgaaaa

SEQ ID NO: 80 - DNA, 20 bp, synthetic construct
  source 1..20: mol_type = other DNA
tcctgagctttgtgaaaggt

SEQ ID NO: 81 - DNA, 22 bp, synthetic construct
  source 1..22: mol_type = other DNA
gcagactttggtttagcaaggt

SEQ ID NO: 82 - DNA, 19 bp, synthetic construct
  source 1..19: mol_type = other DNA
acagtgcagcttcaggagc

SEQ ID NO: 83 - DNA, 20 bp, synthetic construct
  source 1..20: mol_type = other DNA
ggatccctttggacgaaagt

SEQ ID NO: 84 - DNA, 20 bp, synthetic construct
  source 1..20: mol_type = other DNA
agcttgccatgcatcaaaat

SEQ ID NO: 85 - DNA, 21 bp, synthetic construct
  source 1..21: mol_type = other DNA
gaattcctgtgcatgaaagca

SEQ ID NO: 86 - DNA, 24 bp, synthetic construct
  source 1..24: mol_type = other DNA
ttgatgtagttcattcgaagttca

SEQ ID NO: 87 - DNA, 17 bp, synthetic construct
  source 1..17: mol_type = other DNA
cctgtgagggctgcaag

SEQ ID NO: 88 - DNA, 24 bp, synthetic construct
  source 1..24: mol_type = other DNA
tcttcctcctgtttttatcaatgg

SEQ ID NO: 89 - DNA, 20 bp, synthetic construct
  source 1..20: mol_type = other DNA
aagttggccgacaaggagtt

SEQ ID NO: 90 - DNA, 20 bp, synthetic construct
  source 1..20: mol_type = other DNA
acaggctgagctccacaaag

SEQ ID NO: 91 - DNA, 20 bp, synthetic construct
  source 1..20: mol_type = other DNA
gagcccaagaccgagaccta

SEQ ID NO: 92 - DNA, 19 bp, synthetic construct
  source 1..19: mol_type = other DNA
agctgtttgtcggctgctt

SEQ ID NO: 93 - DNA, 21 bp, synthetic construct
  source 1..21: mol_type = other DNA
agcccccagattaactcaaca

SEQ ID NO: 94 - DNA, 20 bp, synthetic construct
  source 1..20: mol_type = other DNA
gattgcacatagccgtttgc

SEQ ID NO: 95 - DNA, 20 bp, synthetic construct
  source 1..20: mol_type = other DNA
gaagtttcccgcaggctatg

SEQ ID NO: 96 - DNA, 21 bp, synthetic construct
  source 1..21: mol_type = other DNA
tgatgggctcatggatgtaga

SEQ ID NO: 97 - DNA, 22 bp, synthetic construct
  source 1..22: mol_type = other DNA
ttgtactttgagaagggggagt

SEQ ID NO: 98 - DNA, 20 bp, synthetic construct
  source 1..20: mol_type = other DNA
ggtagcatgaggggatacgg

SEQ ID NO: 99 - DNA, 24 bp, synthetic construct
  source 1..24: mol_type = other DNA
aagaggagtttaaaaggctgaaca

SEQ ID NO: 100 - DNA, 19 bp, synthetic construct
  source 1..19: mol_type = other DNA
aaggtggcatcccataggc

SEQ ID NO: 101 - DNA, 20 bp, synthetic construct
  source 1..20: mol_type = other DNA
attggagacttcgggatggc

SEQ ID NO: 102 - DNA, 20 bp, synthetic construct
  source 1..20: mol_type = other DNA
cttaactggcagcatggcac

SEQ ID NO: 103 - DNA, 23 bp, synthetic construct
  source 1..23: mol_type = other DNA
ggccagtggagataacactctaa

SEQ ID NO: 104 - DNA, 23 bp, synthetic construct
  source 1..23: mol_type = other DNA
caccattccccattgtgattata

SEQ ID NO: 105 - DNA, 21 bp, synthetic construct
  source 1..21: mol_type = other DNA
agaagcaccttttcagcacca

SEQ ID NO: 106 - DNA, 23 bp, synthetic construct
  source 1..23: mol_type = other DNA
acactggatatttgagcctggat

SEQ ID NO: 107 - DNA, 20 bp, synthetic construct
  source 1..20: mol_type = other DNA
atgaagagctgacccagccg

SEQ ID NO: 108 - DNA, 20 bp, synthetic construct
  source 1..20: mol_type = other DNA
cctcataggactgggaccct

SEQ ID NO: 109 - DNA, 23 bp, synthetic construct
  source 1..23: mol_type = other DNA
gaacatagcacgtttctctctgg

SEQ ID NO: 110 - DNA, 22 bp, synthetic construct
  source 1..22: mol_type = other DNA
gtatcttgaaggggctcacttg

SEQ ID NO: 111 - DNA, 22 bp, synthetic construct
  source 1..22: mol_type = other DNA
ttcctcttcctcctactcacca

SEQ ID NO: 112 - DNA, 20 bp, synthetic construct
  source 1..20: mol_type = other DNA
ctggtgtcgttttgtcctga

SEQ ID NO: 113 - DNA, 19 bp, synthetic construct
  source 1..19: mol_type = other DNA
ggacatcagagggtcgctt

SEQ ID NO: 114 - DNA, 21 bp, synthetic construct
  source 1..21: mol_type = other DNA
gatgggagtccaggagaaaat

SEQ ID NO: 115 - DNA, 17 bp, synthetic construct
  source 1..17: mol_type = other DNA
gacgctgcgcacaacat

SEQ ID NO: 116 - DNA, 24 bp, synthetic construct
  source 1..24: mol_type = other DNA
ctggtcttcaacttccatttcatc

SEQ ID NO: 117 - DNA, 20 bp, synthetic construct
  source 1..20: mol_type = other DNA
gtacccaccgccatactacc

SEQ ID NO: 118 - DNA, 21 bp, synthetic construct
  source 1..21: mol_type = other DNA
gcattttcacatagacccctg

SEQ ID NO: 119 - DNA, 19 bp, synthetic construct
  source 1..19: mol_type = other DNA
gttcaaggatggcgatcag

SEQ ID NO: 120 - DNA, 21 bp, synthetic construct
  source 1..21: mol_type = other DNA
ggccagcatatcacacaatgt

SEQ ID NO: 121 - DNA, 23 bp, synthetic construct
  source 1..23: mol_type = other DNA
cccaggaaactcctaactttcaa

SEQ ID NO: 122 - DNA, 21 bp, synthetic construct
  source 1..21: mol_type = other DNA
tccctccacttcacagagatg

SEQ ID NO: 123 - DNA, 20 bp, synthetic construct
  source 1..20: mol_type = other DNA
gaacagcagtaccgagcctt

SEQ ID NO: 124 - DNA, 20 bp, synthetic construct
  source 1..20: mol_type = other DNA
acttctcgaacagaccgcag

SEQ ID NO: 125 - DNA, 17 bp, synthetic construct
  source 1..17: mol_type = other DNA
tccttaagcgcagccct

SEQ ID NO: 126 - DNA, 21 bp, synthetic construct
  source 1..21: mol_type = other DNA
aggacatgaaaccagacagga

SEQ ID NO: 127 - DNA, 23 bp, synthetic construct
  source 1..23: mol_type = other DNA
gggcttttatacggctcacagac

SEQ ID NO: 128 - DNA, 20 bp, synthetic construct
  source 1..20: mol_type = other DNA
aatggttctcctccagcctt

SEQ ID NO: 129 - DNA, 20 bp, synthetic construct
  source 1..20: mol_type = other DNA
gacgcaggggagtatacgtg

SEQ ID NO: 130 - DNA, 20 bp, synthetic construct
  source 1..20: mol_type = other DNA
tctcttccagggcttccaga

SEQ ID NO: 131 - DNA, 20 bp, synthetic construct
  source 1..20: mol_type = other DNA
cacgaccaagaagccagact

SEQ ID NO: 132 - DNA, 21 bp, synthetic construct
  source 1..21: mol_type = other DNA
tggactcagccgaaactgtta

SEQ ID NO: 133 - DNA, 19 bp, synthetic construct
  source 1..19: mol_type = other DNA
gaatgcctcccacgaggac

SEQ ID NO: 134 - DNA, 20 bp, synthetic construct
  source 1..20: mol_type = other DNA
gaggatggagcgtctgtcac

SEQ ID NO: 135 - DNA, 22 bp, synthetic construct
  source 1..22: mol_type = other DNA
ccgatgtatggtcatttggaat

SEQ ID NO: 136 - DNA, 19 bp, synthetic construct
  source 1..19: mol_type = other DNA
ctggattacctgggcacct

SEQ ID NO: 137 - DNA, 21 bp, synthetic construct
  source 1..21: mol_type = other DNA
ggttggtcaactggcagaaac

SEQ ID NO: 138 - DNA, 20 bp, synthetic construct
  source 1..20: mol_type = other DNA
agagctcctttcaggtctcg

SEQ ID NO: 139 - DNA, 19 bp, synthetic construct
  source 1..19: mol_type = other DNA
atcttcaccctgacacggg

SEQ ID NO: 140 - DNA, 21 bp, synthetic construct
  source 1..21: mol_type = other DNA
ttagagtcttcagccgtgctc

SEQ ID NO: 141 - DNA, 23 bp, synthetic construct
  source 1..23: mol_type = other DNA
tctgctactactacgacggtgat

SEQ ID NO: 142 - DNA, 20 bp, synthetic construct
  source 1..20: mol_type = other DNA
cagtggctttatggggccta

SEQ ID NO: 143 - DNA, 23 bp, synthetic construct
  source 1..23: mol_type = other DNA
tggcattgatgaccagagttaca

SEQ ID NO: 144 - DNA, 20 bp, synthetic construct
  source 1..20: mol_type = other DNA
tgcacgtgggttggtagaag

SEQ ID NO: 145 - DNA, 20 bp, synthetic construct
  source 1..20: mol_type = other DNA
ctggcgtcgtgattagtgat

SEQ ID NO: 146 - DNA, 21 bp, synthetic construct
  source 1..21: mol_type = other DNA
gagcaagacgttcagtcctgt

SEQ ID NO: 147 - DNA, 21 bp, synthetic construct
  source 1..21: mol_type = other DNA
tgctgcgaaagtcttcttgag

SEQ ID NO: 148 - DNA, 24 bp, synthetic construct
  source 1..24: mol_type = other DNA
tggctgttcagagaaatactcatc

SEQ ID NO: 149 - DNA, 20 bp, synthetic construct
  source 1..20: mol_type = other DNA
tgagccagaatgccttcatc

SEQ ID NO: 150 - DNA, 23 bp, synthetic construct
  source 1..23: mol_type = other DNA
agattcatctgtgtaatcaggcg

SEQ ID NO: 151 - DNA, 21 bp, synthetic construct
  source 1..21: mol_type = other DNA
gaatgcaagagaggtttccgc

SEQ ID NO: 152 - DNA, 20 bp, synthetic construct
  source 1..20: mol_type = other DNA
tgttccgagtggcagagctt

SEQ ID NO: 153 - DNA, 19 bp, synthetic construct
  source 1..19: mol_type = other DNA
acacttcctgccaagtcca

SEQ ID NO: 154 - DNA, 18 bp, synthetic construct
  source 1..18: mol_type = other DNA
atgcttgactcacgggga

SEQ ID NO: 155 - DNA, 20 bp, synthetic construct
  source 1..20: mol_type = other DNA
atttctggctggaacggacg

SEQ ID NO: 156 - DNA, 20 bp, synthetic construct
  source 1..20: mol_type = other DNA
gccagtcccttagacacacc

SEQ ID NO: 157 - DNA, 23 bp, synthetic construct
  source 1..23: mol_type = other DNA
cctcctactcctgaagacaacag

SEQ ID NO: 158 - DNA, 23 bp, synthetic construct
  source 1..23: mol_type = other DNA
tgaggatcattggtttggtagtc

SEQ ID NO: 159 - DNA, 20 bp, synthetic construct
  source 1..20: mol_type = other DNA
cacaggcatgccgtatctct

SEQ ID NO: 160 - DNA, 20 bp, synthetic construct
  source 1..20: mol_type = other DNA
ccagagcttggtgttctcgt

SEQ ID NO: 161 - DNA, 24 bp, synthetic construct
  source 1..24: mol_type = other DNA
tgctgcttctaaagcttgtggtat

SEQ ID NO: 162 - DNA, 21 bp, synthetic construct
  source 1..21: mol_type = other DNA
ggaagacatggttgggtggat

SEQ ID NO: 163 - DNA, 22 bp, synthetic construct
  source 1..22: mol_type = other DNA
tgacaatgtctcaccaagctct

SEQ ID NO: 164 - DNA, 20 bp, synthetic construct
  source 1..20: mol_type = other DNA
gaggacaatggacagagccc

SEQ ID NO: 165 - DNA, 21 bp, synthetic construct
  source 1..21: mol_type = other DNA
ccatctcaagctttcagagga

SEQ ID NO: 166 - DNA, 22 bp, synthetic construct
  source 1..22: mol_type = other DNA
tagcttctatcctttccaggga

SEQ ID NO: 167 - DNA, 20 bp, synthetic construct
  source 1..20: mol_type = other DNA
tgacacaccgtgagacaggt

SEQ ID NO: 168 - DNA, 21 bp, synthetic construct
  source 1..21: mol_type = other DNA
gcatgaccttcacctccttga

SEQ ID NO: 169 - DNA, 21 bp, synthetic construct
  source 1..21: mol_type = other DNA
gggagaatcttcaggtgactc

SEQ ID NO: 170 - DNA, 20 bp, synthetic construct
  source 1..20: mol_type = other DNA
ccaggaccaggacattcagt

SEQ ID NO: 171 - DNA, 19 bp, synthetic construct
  source 1..19: mol_type = other DNA
ccatcacctgcctcagaac

SEQ ID NO: 172 - DNA, 20 bp, synthetic construct
  source 1..20: mol_type = other DNA
aatacactgggagccagagc

SEQ ID NO: 173 - DNA, 19 bp, synthetic construct
  source 1..19: mol_type = other DNA
agctctgcggagaccaact

SEQ ID NO: 174 - DNA, 20 bp, synthetic construct
  source 1..20: mol_type = other DNA
gctgctgctcatcaagctct

SEQ ID NO: 175 - DNA, 23 bp, synthetic construct
  source 1..23: mol_type = other DNA
tatattgtgaacgagccacctcc

SEQ ID NO: 176 - DNA, 22 bp, synthetic construct
  source 1..22: mol_type = other DNA
tggtttgtgagcatcttcaggt

SEQ ID NO: 177 - DNA, 21 bp, synthetic construct
  source 1..21: mol_type = other DNA
cgaggacttcagcgaagtgta

SEQ ID NO: 178 - DNA, 20 bp, synthetic construct
  source 1..20: mol_type = other DNA
gccgagtggatgtacttcag

SEQ ID NO: 179 - DNA, 22 bp, synthetic construct
  source 1..22: mol_type = other DNA
gagcactgctttaataggacac

SEQ ID NO: 180 - DNA, 20 bp, synthetic construct
  source 1..20: mol_type = other DNA
gttcgatattcatcacggcg

SEQ ID NO: 181 - DNA, 23 bp, synthetic construct
  source 1..23: mol_type = other DNA
ccaaacgaggagaggaattaaag

SEQ ID NO: 182 - DNA, 24 bp, synthetic construct
  source 1..24: mol_type = other DNA
gcctgtacagtttcatttggattt

SEQ ID NO: 183 - DNA, 24 bp, synthetic construct
  source 1..24: mol_type = other DNA
aagttttcttcaaaagagcagtgg

SEQ ID NO: 184 - DNA, 20 bp, synthetic construct
  source 1..20: mol_type = other DNA
tcgatgatgcaatcattcct

SEQ ID NO: 185 - DNA, 19 bp, synthetic construct
  source 1..19: mol_type = other DNA
tctggagctccgtgatctg

SEQ ID NO: 186 - DNA, 21 bp, synthetic construct
  source 1..21: mol_type = other DNA
agtgaaatggaaggcatctgg

SEQ ID NO: 187 - DNA, 20 bp, synthetic construct
  source 1..20: mol_type = other DNA
actttgctgggatcctgtcc

SEQ ID NO: 188 - DNA, 21 bp, synthetic construct
  source 1..21: mol_type = other DNA
caaacatgtagcctgtcacgg

SEQ ID NO: 189 - DNA, 21 bp, synthetic construct
  source 1..21: mol_type = other DNA
tcacaggttacatgtttggga

SEQ ID NO: 190 - DNA, 25 bp, synthetic construct
  source 1..25: mol_type = other DNA
ttcattacactgacctagagctacc

SEQ ID NO: 191 - DNA, 20 bp, synthetic construct
  source 1..20: mol_type = other DNA
caagaacaactgggcagagc

SEQ ID NO: 192 - DNA, 19 bp, synthetic construct
  source 1..19: mol_type = other DNA
ggcctctgtccaccttcac

SEQ ID NO: 193 - DNA, 16 bp, synthetic construct
  source 1..16: mol_type = other DNA
ataggagccaggcgca

SEQ ID NO: 194 - DNA, 18 bp, synthetic construct
  source 1..18: mol_type = other DNA
ccagctccccatagtcca

SEQ ID NO: 195 - DNA, 20 bp, synthetic construct
  source 1..20: mol_type = other DNA
gagctgacgttctctcagca

SEQ ID NO: 196 - DNA, 19 bp, synthetic construct
  source 1..19: mol_type = other DNA
tacctccggggaacagcat

SEQ ID NO: 197 - DNA, 22 bp, synthetic construct
  source 1..22: mol_type = other DNA
caggcatgagtacctgtatggc

SEQ ID NO: 198 - DNA, 18 bp, synthetic construct
  source 1..18: mol_type = other DNA
catggtcaggtgaggggt

SEQ ID NO: 199 - DNA, 20 bp, synthetic construct
  source 1..20: mol_type = other DNA
tggttcacgctcctgtatga

SEQ ID NO: 200 - DNA, 18 bp, synthetic construct
  source 1..18: mol_type = other DNA
ctctgtttgctgcccagc

SEQ ID NO: 201 - DNA, 18 bp, synthetic construct
  source 1..18: mol_type = other DNA
acggtcactcgcatcctg

SEQ ID NO: 202 - DNA, 20 bp, synthetic construct
  source 1..20: mol_type = other DNA
ggtccagtcaggtctacgcc

SEQ ID NO: 203 - DNA, 20 bp, synthetic construct
  source 1..20: mol_type = other DNA
caattcatacgcgggatagc

SEQ ID NO: 204 - DNA, 18 bp, synthetic construct
  source 1..18: mol_type = other DNA
ccatgaaaaccgctgcat

SEQ ID NO: 205 - DNA, 17 bp, synthetic construct
  source 1..17: mol_type = other DNA
tcacacgccgaaacctg

SEQ ID NO: 206 - DNA, 21 bp, synthetic construct
  source 1..21: mol_type = other DNA
ggcccttcatgctcatcatag

SEQ ID NO: 207 - DNA, 18 bp, synthetic construct
  source 1..18: mol_type = other DNA
gcagcagtggattctcgg

SEQ ID NO: 208 - DNA, 24 bp, synthetic construct
  source 1..24: mol_type = other DNA
gcaggtaagggttaatctcaatca

SEQ ID NO: 209 - DNA, 18 bp, synthetic construct
  source 1..18: mol_type = other DNA
catggtagctggctggga

SEQ ID NO: 210 - DNA, 20 bp, synthetic construct
  source 1..20: mol_type = other DNA
aaggctgtcgagtcagcatt

SEQ ID NO: 211 - DNA, 23 bp, synthetic construct
  source 1..23: mol_type = other DNA
gggacctcttcaatgtcaatgat

SEQ ID NO: 212 - DNA, 23 bp, synthetic construct
  source 1..23: mol_type = other DNA
tccacacattgtttggtgtagtg

SEQ ID NO: 213 - DNA, 22 bp, synthetic construct
  source 1..22: mol_type = other DNA
ggtgcctgatgacatctacaaa

SEQ ID NO: 214 - DNA, 19 bp, synthetic construct
  source 1..19: mol_type = other DNA
gcagagtccacctgagagc

SEQ ID NO: 215 - DNA, 20 bp, synthetic construct
  source 1..20: mol_type = other DNA
gatcagggtcagcgagaagc

SEQ ID NO: 216 - DNA, 20 bp, synthetic construct
  source 1..20: mol_type = other DNA
ccgccagatcttgtagtgcc

SEQ ID NO: 217 - DNA, 20 bp, synthetic construct
  source 1..20: mol_type = other DNA
ttagcctgccctctttggac

SEQ ID NO: 218 - DNA, 20 bp, synthetic construct
  source 1..20: mol_type = other DNA
tcagaagggaggcagacagt

SEQ ID NO: 219 - DNA, 20 bp, synthetic construct
  source 1..20: mol_type = other DNA
agcaagcctcacatgtttcc

SEQ ID NO: 220 - DNA, 21 bp, synthetic construct
  source 1..21: mol_type = other DNA
caaggtaattacacgctctgc

SEQ ID NO: 221 - DNA, 20 bp, synthetic construct
  source 1..20: mol_type = other DNA
agctcatcaccaaggtcagc

SEQ ID NO: 222 - DNA, 20 bp, synthetic construct
  source 1..20: mol_type = other DNA
tcgtggtatacttgcccagc

SEQ ID NO: 223 - DNA, 21 bp, synthetic construct
  source 1..21: mol_type = other DNA
tactgccaagtcatccgtgtc

SEQ ID NO: 224 - DNA, 20 bp, synthetic construct
  source 1..20: mol_type = other DNA
tctccatcaggtgggccttc

SEQ ID NO: 225 - DNA, 22 bp, synthetic construct
  source 1..22: mol_type = other DNA
cctgatgctcatggagtgttcc

SEQ ID NO: 226 - DNA, 22 bp, synthetic construct
  source 1..22: mol_type = other DNA
ggaaagtggaggtgctgcacac

SEQ ID NO: 227 - DNA, 21 bp, synthetic construct
  source 1..21: mol_type = other DNA
ttctgctccctgtcagcttcc

SEQ ID NO: 228 - DNA, 21 bp, synthetic construct
  source 1..21: mol_type = other DNA
tcccagaaaccctttggtccg

SEQ ID NO: 229 - DNA, 22 bp, synthetic construct
  source 1..22: mol_type = other DNA
atgtcagtgggacttgtgctgc

SEQ ID NO: 230 - DNA, 22 bp, synthetic construct
  source 1..22: mol_type = other DNA
aactcagccaccatgaaccagg

SEQ ID NO: 231 - DNA, 19 bp, synthetic construct
  source 1..19: mol_type = other DNA
gaagtgcccttggttcgga

SEQ ID NO: 232 - DNA, 20 bp, synthetic construct
  source 1..20: mol_type = other DNA
gcagggtagcgattcgagtt

SEQ ID NO: 233 - DNA, 22 bp, synthetic construct
  source 1..22: mol_type = other DNA
actcaatggaagccacacatgg

SEQ ID NO: 234 - DNA, 22 bp, synthetic construct
  source 1..22: mol_type = other DNA
tacggctctgtctggtttgagg

SEQ ID NO: 235 - DNA, 20 bp, synthetic construct
  source 1..20: mol_type = other DNA
cgtggtcatcctctcctacg

SEQ ID NO: 236 - DNA, 19 bp, synthetic construct
  source 1..19: mol_type = other DNA
accttctggaagctctggc

SEQ ID NO: 237 - DNA, 23 bp, synthetic construct
  source 1..23: mol_type = other DNA
gttggcctttctgatcatattgc

SEQ ID NO: 238 - DNA, 20 bp, synthetic construct
  source 1..20: mol_type = other DNA
ctgcacagctggttcttccc

SEQ ID NO: 239 - DNA, 22 bp, synthetic construct
  source 1..22: mol_type = other DNA
atgttccttcagtcgtcaatgc

SEQ ID NO: 240 - DNA, 21 bp, synthetic construct
  source 1..21: mol_type = other DNA
ttgctgcactctgcaataact

SEQ ID NO: 241 - DNA, 22 bp, synthetic construct
  source 1..22: mol_type = other DNA
atctgtgccacatcagccacca

SEQ ID NO: 242 - DNA, 22 bp, synthetic construct
  source 1..22: mol_type = other DNA
aaggtggtgtccttctcagcca

SEQ ID NO: 243 - DNA, 22 bp, synthetic construct
  source 1..22: mol_type = other DNA
cagcacatcagagcagagaaag

SEQ ID NO: 244 - DNA, 20 bp, synthetic construct
  source 1..20: mol_type = other DNA
agcaaaaggctgagcttcaa

SEQ ID NO: 245 - DNA, 20 bp, synthetic construct
  source 1..20: mol_type = other DNA
cagggaggagaagcagaagc

SEQ ID NO: 246 - DNA, 20 bp, synthetic construct
  source 1..20: mol_type = other DNA
aggcggctcaatcttgaagt

SEQ ID NO: 247 - DNA, 22 bp, synthetic construct
  source 1..22: mol_type = other DNA
atgtcagacgaatgctagatgg

SEQ ID NO: 248 - DNA, 20 bp, synthetic construct
  source 1..20: mol_type = other DNA
ccaagttcttgaatcgtgcc

SEQ ID NO: 249 - DNA, 20 bp, synthetic construct
  source 1..20: mol_type = other DNA
gcgttttggaaagataccca

SEQ ID NO: 250 - DNA, 18 bp, synthetic construct
  source 1..18: mol_type = other DNA
ttgccaatctggacacca

SEQ ID NO: 251 - DNA, 23 bp, synthetic construct
  source 1..23: mol_type = other DNA
agctctattgcttggaacatggg

SEQ ID NO: 252 - DNA, 21 bp, synthetic construct
  source 1..21: mol_type = other DNA
cccctccaccaatggtcttgt

SEQ ID NO: 253 - DNA, 23 bp, synthetic construct
  source 1..23: mol_type = other DNA
gcgtgaaattgtccatattcaga

SEQ ID NO: 254 - DNA, 22 bp, synthetic construct
  source 1..22: mol_type = other DNA
aatcatctcccagaacttggct

SEQ ID NO: 255 - DNA, 24 bp, synthetic construct
  source 1..24: mol_type = other DNA
gcctgacaatttcatctttggtca

SEQ ID NO: 256 - DNA, 20 bp, synthetic construct
  source 1..20: mol_type = other DNA
caggcagtcgcagttttcac

SEQ ID NO: 257 - DNA, 18 bp, synthetic construct
  source 1..18: mol_type = other DNA
cagctggaccgcatctct

SEQ ID NO: 258 - DNA, 18 bp, synthetic construct
  source 1..18: mol_type = other DNA
agatccaccaggatggca

SEQ ID NO: 259 - DNA, 22 bp, synthetic construct
  source 1..22: mol_type = other DNA
atatgattgttgggaaggcatg

SEQ ID NO: 260 - DNA, 22 bp, synthetic construct
  source 1..22: mol_type = other DNA
aagtcctcttcttcgattccaa

SEQ ID NO: 261 - DNA, 20 bp, synthetic construct
  source 1..20: mol_type = other DNA
ttctgggacctggcactaag

SEQ ID NO: 262 - DNA, 21 bp, synthetic construct
  source 1..21: mol_type = other DNA
atcaccaaccactctggtgtc

SEQ ID NO: 263 - DNA, 22 bp, synthetic construct
  source 1..22: mol_type = other DNA
ggagaaaagatccatgaggaca

SEQ ID NO: 264 - DNA, 18 bp, synthetic construct
  source 1..18: mol_type = other DNA
cccagcaatggagtgaca

SEQ ID NO: 265 - DNA, 20 bp, synthetic construct
  source 1..20: mol_type = other DNA
cttgccttgctgctctacct

SEQ ID NO: 266 - DNA, 22 bp, synthetic construct
  source 1..22: mol_type = other DNA
tccatgaacttcaccacttcgt

SEQ ID NO: 267 - DNA, 19 bp, synthetic construct
  source 1..19: mol_type = other DNA
ctgacgatggcctggagtg

SEQ ID NO: 268 - DNA, 20 bp, synthetic construct
  source 1..20: mol_type = other DNA
cgggtaccggatcatgagga

(19) United States
(12) Patent Application Publication
(10) Pub. No.: US 2025/0092467 A1
(43) Pub. Date: Mar. 20, 2025

(54) CUSTOMIZED CANCER TREATMENT METHOD FOR CANCER PATIENTS USING DIGITAL PCR

(71) Applicant: ONCOin Co., Ltd., Chuncheon-si (KR)

(72) Inventor: Yang Sik JEONG, Wonju-si Gangwon-do (KR)

(21) Appl. No.: 18/964,177

(22) Filed: Nov. 29, 2024

Related U.S. Application Data
(63) Continuation of application No. PCT/KR2023/007248, filed on May 26, 2023.

(30) Foreign Application Priority Data
May 30, 2022 (KR) 10-2022-0066121

(51) Int. Cl. C12Q 1/6886; C12Q 1/6851

(52) U.S. Cl. CPC C12Q 1/6886; C12Q 1/6851; C12Q 2600/106; C12Q 2600/158

(57) ABSTRACT

The present invention relates to a method for personalized cancer treatment for cancer patients using digital PCR. The results of quantitative analysis of expression changes in 134 target genes using digital PCR have confirmed that differences in the expression patterns of target genes among cancer patients are present. Furthermore, it has been established that this method allows for simpler and more accurate analysis of expression patterns compared to qPCR. Therefore, the method for analyzing target gene expression patterns in cancer patients according to the present invention can provide information enables the selective administration of existing or future-developed cancer therapeutic agents to individual cancer patients, thereby facilitating personalized treatment for each patient.

### CROSS-REFERENCE TO RELATED APPLICATIONS

This application is a continuation of International Application No. PCT/KR2023/007248 filed 26 May 2023, which claims the benefit of, and relies on the filing date of, Korean Patent Application No. 10-2022-0066121 filed 30 May 2022, the entire disclosure of which is incorporated herein by reference.

### SEQUENCE LISTING

The instant application contains a Sequence Listing which has been submitted electronically in XML format and is hereby incorporated by reference in its entirety. Said XML copy, created on 29 Nov. 2024, is named SL_0363_0044-PCT-US.xml and is 237, 105 bytes in size.

### TECHNICAL FIELD

The present invention relates to a method for personalized cancer treatment for cancer patients using digital PCR, and more specifically, to a method for screening personalized therapeutic agents for cancer patients using digital PCR and a kit used therefor.

### BACKGROUND ART

Cancer is a disease that poses a threat to human health and life, accounting for approximately 13% of all deaths. In 2007, 7.6 million people worldwide died from cancer. In the United States, an estimated 1.4 million new cases of cancer have been reported annually over recent years, and cancer ranks as the second leading cause of death. According to statistics from the SEER (Surveillance, Epidemiology, and End Results) report, the mortality rate for all types of cancer in the United States increased from 195.4 per 100,000 in 1950 to 204.4 per 100,000 in 1978, and then steadily decreased to 184.0 per 100,000 by 2005. This downward trend is thought to result from the early detection of cancer made possible by improved diagnostic technologies. Early detection and treatment play an essential role in the prognosis and survival of all types of cancer. There are approximately 300 cancer treatments that have been approved for clinical use by the U.S. FDA (Food and Drug Administration) and the European EMA (European Medicines Agency). These cancer treatments have been approved for indications for at least one type of cancer, targeting specific biomarkers.

According to conventional cancer chemotherapy, suitable anticancer agents are selected and administered based on the type and severity of cancer rather than tailored to the individual cancer patient. However, clinical results generally indicate that the therapeutic efficacy of such chemotherapy varies significantly among patients, and various methods have been proposed to overcome this limitation.

Meanwhile, the digital PCR method, which enables absolute quantification with a small amount of genetic sample, has recently gained attention and is in the stage of technological optimization for application in molecular diagnostics. The advantage of digital PCR, allowing absolute quantification with as little as several picograms (pg) or nanograms (ng) of genetic material, makes it highly suitable for determining gene copy numbers. Consequently, copy number analysis methods using digital PCR are being continuously developed for various disease-related genes (KR Patent Publication No. 10-2017-0051256).

Accordingly, as a result of efforts to develop a simple and accurate method for selecting therapeutic agents suitable for individual cancer patients, the present inventors have discovered that it is possible to profile expression patterns specific to each cancer patient with high accuracy and convenience by quantifying and analyzing expression changes in 134 genes targeted by FDA-approved cancer therapies using the digital PCR method. This enables the selection of personalized therapeutic agents for cancer patients, leading to the present application.

### DETAILED DESCRIPTION OF THE INVENTION

### Technical Problem

An object of the present invention is to provide a method for more efficiently conducting chemotherapy for cancer patients, specifically offering a kit for screening personalized therapeutic agents for cancer patients using cancer therapeutic targets, and the use thereof.

### Solution to Problem

To achieve the object of the present invention, the present invention provides a kit for screening personalized therapeutic agents for cancer patients, comprising primers or probes that specifically hybridize to the target genes ADA and CD52; and primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, Lck, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ.

In addition, the present invention provides a method for screening personalized therapeutic agents for cancer patients, comprising the steps of:
1) measuring the expression of the target genes in a biological sample isolated from a cancer patient, wherein the target genes comprise ADA and CD52 essentially; and at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, Lck, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ; and
2) screening for drugs that act on the target genes measured to be highly expressed in step 1).

The present invention further provides a method for screening personalized therapeutic agents for cancer patients, comprising the following steps:
1) isolating and proliferating cancer cells and normal cells from cancer tissue and normal tissue obtained from a cancer patient;
2) treating the cancer cells from step 1) with a candidate anticancer agent;
3) isolating total RNA from the cancer cells and normal cells of step 1), and from the cancer cells treated with the candidate anticancer agent of step 2), and synthesizing cDNA using the total RNA as a template;
4) using the synthesized cDNA of step 3) as a template, generating multiple droplets from a reaction solution containing primers or probes that specifically hybridize to the target genes ADA and CD52, as well as primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ;
5) amplifying the target nucleic acid sequences in the droplets generated in step 4); and
6) counting the number of droplets containing the amplified target nucleic acids from step 5) to determine the amount of amplified target nucleic acid, followed by comparing the amount of amplified target nucleic acid in normal cells, cancer cells, and cancer cells treated with the candidate anticancer agent.

### Advantageous Effects of Invention

The present invention quantifies expression changes of 134 cancer therapeutic target genes between normal and cancer tissues in cancer patients using digital PCR and provides a simple and accurate analysis to confirm differences in target gene expression patterns among cancer patients. Thus, the present invention suggests that it can provide information for screening personalized therapeutic agents for cancer patients. Accordingly, the present invention can be utilized for personalized treatment by selectively administering existing or future-developed cancer therapeutic agents to individual cancer patients.

### BRIEF DESCRIPTION OF DRAWINGS

FIGS. 1a to 1e illustrate the expression patterns of 134 target genes obtained by performing qPCR on normal tissue and cancer tissue samples collected from three renal cancer patients, according to one embodiment of the present invention. The first N and T represent the normal tissue (N) and cancer tissue (T) samples from Renal Cancer Patient 1, the second N and T represent the normal tissue (N) and cancer tissue (T) samples from Renal Cancer Patient 2, and the third N and T represent the normal tissue (N) and cancer tissue (T) samples from Renal Cancer Patient 3.

FIG. 2 illustrates the expression patterns of 134 target genes obtained by performing ddPCR on normal tissue and cancer tissue samples collected from three renal cancer patients, according to one embodiment of the present invention. The first N and T represent the normal tissue (N) and cancer tissue (T) samples from Renal Cancer Patient 1, the second N and T represent the normal tissue (N) and cancer tissue (T) samples from Renal Cancer Patient 2, and the third N and T represent the normal tissue (N) and cancer tissue (T) samples from Renal Cancer Patient 3

FIG. 3 illustrates the expression patterns of 134 target genes obtained by performing ddPCR on normal tissue and cancer tissue samples collected from Renal Cancer Patient 3, according to one embodiment of the present invention. Here, N represents the normal tissue sample, and T represents the cancer tissue sample.

### BEST MODE FOR CARRYING OUT THE INVENTION

The present invention is described in further detail below.

The present invention provides a kit for screening personalized therapeutic agents for cancer patients, comprising primers or probes that specifically hybridize to the target genes ADA and CD52; and primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ.

In the present invention, the targets essentially include ADA and CD52, and may further comprise at least 10, 15, 20, 21, 22, 23, 24, 25, 26, 27, 28, 29, 30, 31, 32, 33, 34, 35, 36, 37, 38, 39, 40, 41, 42, 43, 44, or 45 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ, or may comprise all of the aforementioned targets, but is not limited thereto.

Additionally, the present invention provides a kit for screening personalized therapeutic agents for cancer patients, further comprising primers or probes that specifically hybridize to at least 10 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

In the present invention, the targets may comprise at least 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 targets selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA and VEGFB, or may comprise all of the aforementioned targets, but is not limited thereto.

In the present invention, the kit may be a digital PCR kit, specifically, a droplet digital PCR kit.

The present terminology, “digital PCR (Digital polymerase chain reaction)”, refers to a novel approach for detecting and quantifying nucleic acids, which allows for more precise quantification and highly sensitive detection of target nucleic acid molecules compared to conventional qPCR. While conventional qPCR utilizes an analog-based approach for result analysis, digital PCR employs a digital method wherein the signal yields a value of either “0” or “1,” allowing for the analysis of large sample volumes, simultaneous testing of multiple samples, and the execution of various test items concurrently. Digital PCR technology enables absolute quantification by applying a single-molecule counting method without requiring a standard curve for DNA samples. This technology conducts PCR reactions on a one droplet per well basis, enabling more accurate absolute quantification.

The present terminology, “primer,” refers to a short nucleic acid sequence possessing a free 3′ hydroxyl group, capable of forming base pairs with a complementary template and serving as an initiation point for template strand replication. In other words, a primer is a single-stranded oligonucleotide that can initiate template-directed DNA synthesis under appropriate conditions in a suitable buffer (for example, in the presence of four different nucleoside triphosphates, DNA, and a polymerase enzyme such as DNA polymerase) and at an appropriate temperature.

The primer of the present invention can be chemically synthesized using the phosphoramidite solid support method or other widely known methods. Additionally, this primer may be modified (e.g., addition, deletion, substitution) using numerous techniques well-known in the art, provided such modifications do not interfere with the detection of the target gene. While it is not necessary for the primer to be completely complementary to the template, it must be sufficiently complementary to allow hybridization with the template. Non-limiting examples of such modifications include methylation, capping, substitution with one or more analogs of natural nucleotides, and modification of inter-nucleotide linkages. Examples of such linkages include uncharged linkages (e.g., methyl phosphonate, phosphotriester, phosphoroamidate, carbamate) and charged linkages (e.g., phosphorothioate, phosphorodithioate). The nucleic acid may also contain one or more additional covalently attached moieties, such as proteins (e.g., nucleases, toxins, antibodies, signal peptides, poly-L-lysine), intercalators (e.g., acridine, proflavine), chelating agents (e.g., metals, radioactive metals, iron, oxidative metals), and alkylating agents. The nucleic acid sequence of the present invention may further be modified with labels capable of directly or indirectly providing a detectable signal, with examples of such labels including radioactive isotopes, fluorescent molecules, and biotin.

The present terminology, “probe,” refers to a linear oligomer of natural or modified monomers or linkages, including deoxyribonucleotides and ribonucleotides, which is capable of specifically hybridizing to a target nucleotide sequence and may exist naturally or be artificially synthesized.

The nucleotide sequence of the target of the present invention, which should be referenced in the production of the primer or probe, can be found in GenBank, and this sequence may be used as a reference for designing the primer or probe.

For example, primer sets that specifically hybridize to each target gene of the present invention, as shown in [Table 1] and [Table 2], may be utilized, and specifically, these may be used for digital PCR, and more specifically for droplet digital PCR.

Specifically, primers that specifically hybridize to each of the target genes ADA and CD52 may be selected from the group consisting of primers designated by sequence numbers 99, 100, 111, and 112 in [Table 2]; primers that specifically hybridize to each of the target genes ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ may be selected from the group consisting of primers designated by sequence numbers 1 to 96 in [Table 1]. More specifically, primers that specifically hybridize to each of the target genes ADA and CD52 may be primer sets consisting of the nucleotide sequences of sequence numbers 99 and 100; and the nucleotide sequences of sequence numbers 111 and 112, respectively. Primers that specifically hybridize to each of the target genes ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ may be primer sets consisting of the nucleotide sequences of sequence numbers 1 and 2; sequence numbers 3 and 4; sequence numbers 5 and 6; sequence numbers 7 and 8; sequence numbers 9 and 10; sequence numbers 11 and 12; sequence numbers 13 and 14; sequence numbers 15 and 16; sequence numbers 17 and 18; sequence numbers 19 and 20; sequence numbers 21 and 22; sequence numbers 23 and 24; sequence numbers 25 and 26; sequence numbers 27 and 28; sequence numbers 29 and 30; sequence numbers 31 and 32; sequence numbers 33 and 34; sequence numbers 35 and 36; sequence numbers 37 and 38; sequence numbers 39 and 40; sequence numbers 41 and 42; sequence numbers 43 and 44; sequence numbers 45 and 46; sequence numbers 47 and 48; sequence numbers 49 and 50; sequence numbers 51 and 52; sequence numbers 53 and 54; sequence numbers 55 and 56; sequence numbers 57 and 58; sequence numbers 59 and 60; sequence numbers 61 and 62; sequence numbers 63 and 64; sequence numbers 65 and 66; sequence numbers 67 and 68; sequence numbers 69 and 70; sequence numbers 71 and 72; sequence numbers 73 and 74; sequence numbers 75 and 76; sequence numbers 77 and 78; sequence numbers 79 and 80; sequence numbers 81 and 82; sequence numbers 83 and 84; sequence numbers 85 and 86; sequence numbers 87 and 88; sequence numbers 89 and 90; sequence numbers 91 and 92; sequence numbers 93 and 94; and sequence numbers 95 and 96.

Additionally, primers that specifically hybridize to each of the target genes ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB may be selected from the group consisting of primers designated by sequence the nucleotide sequences of sequence numbers 97, 98, 101 to 110, and 113 to 268 in [Table 2]. More specifically, primers that specifically hybridize to each of the target genes ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB may be primer sets consisting of the nucleotide sequences of sequence numbers 97 and 98; sequence numbers 101 and 102; sequence numbers 103 and 104; sequence numbers 105 and 106; sequence numbers 107 and 108; sequence numbers 109 and 110; sequence numbers 113 and 114; sequence numbers 115 and 116; sequence numbers 117 and 118; sequence numbers 119 and 120; sequence numbers 121 and 122; sequence numbers 123 and 124; sequence numbers 125 and 126; sequence numbers 127 and 128; sequence numbers 129 and 130; sequence numbers 131 and 132; sequence numbers 133 and 134; sequence numbers 135 and 136; sequence numbers 137 and 138; sequence numbers 139 and 140; sequence numbers 141 and 142; sequence numbers 143 and 144; sequence numbers 145 and 146; sequence numbers 147 and 148; sequence numbers 149 and 150; sequence numbers 151 and 152; sequence numbers 153 and 154; sequence numbers 155 and 156; sequence numbers 157 and 158; sequence numbers 159 and 160; sequence numbers 161 and 162; sequence numbers 163 and 164; sequence numbers 165 and 166; sequence numbers 167 and 168; sequence numbers 169 and 170; sequence numbers 171 and 172; sequence numbers 173 and 174; sequence numbers 175 and 176; sequence numbers 177 and 178; sequence numbers 179 and 180; sequence numbers 181 and 182; sequence numbers 183 and 184; sequence numbers 185 and 186; sequence numbers 187 and 188; sequence numbers 189 and 190; sequence numbers 191 and 192; sequence numbers 193 and 194; sequence numbers 195 and 196; sequence numbers 197 and 198; sequence numbers 199 and 200; sequence numbers 201 and 202; sequence numbers 203 and 204; sequence numbers 205 and 206; sequence numbers 207 and 208; sequence numbers 209 and 210; sequence numbers 211 and 212; sequence numbers 213 and 214; sequence numbers 215 and 216; sequence numbers 217 and 218; sequence numbers 219 and 220; sequence numbers 221 and 222; sequence numbers 223 and 224; sequence numbers 225 and 226; sequence numbers 227 and 228; sequence numbers 229 and 230; sequence numbers 231 and 232; sequence numbers 233 and 234; sequence numbers 235 and 236; sequence numbers 237 and 238; sequence numbers 239 and 240; sequence numbers 241 and 242; sequence numbers 243 and 244; sequence numbers 245 and 246; sequence numbers 247 and 248; sequence numbers 249 and 250; sequence numbers 251 and 252; sequence numbers 253 and 254; sequence numbers 255 and 256; sequence numbers 257 and 258; sequence numbers 259 and 260; sequence numbers 261 and 262; sequence numbers 263 and 264; sequence numbers 265 and 266; and sequence numbers 267 and 268.

In the present invention, the cancer may be renal cancer, gastric cancer, lung cancer, breast cancer, ovarian cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer, ureter cancer, or hematological cancer, and may specifically be renal cancer, but is not limited thereto.

Additionally, the cancer patient may exhibit symptoms of advanced cancer, regardless of the type of cancer, such as ascites or pleural effusion, but is not limited thereto.

In the present invention, the kit may include DNA polymerase, dNTPs, buffers, and the like for performing PCR amplification reactions. The kit may also further include a user guide detailing the optimal reaction conditions. The guide is a printed document that describes the use of the kit, such as the preparation methods for reverse transcription buffer and PCR buffer, and the presented reaction conditions. The guide may be in the form of a pamphlet or leaflet, a label attached to the kit, or descriptions included on the surface of the package containing the kit. Additionally, the guide may include information disclosed or provided through electronic media, such as the internet.

In the present invention, the expression changes of 134 cancer therapeutic target genes between the normal and cancer tissues of cancer patients were quantified using digital PCR, revealing differences in the expression patterns of cancer therapeutic targets among individual cancer patients. Furthermore, it was confirmed that digital PCR enables more accurate analysis compared to qPCR. Therefore, the expression profiling of the 134 cancer therapeutic target genes through digital PCR according to the present invention can be utilized for the selection of personalized cancer therapies for cancer patients.

Additionally, the present invention provides a method for screening personalized therapeutic agents for cancer patients, comprising the steps of:
1) measuring the expression of the target genes in a biological sample isolated from a cancer patient wherein the target genes comprise ADA and CD52 essentially; and at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, Lck, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ; and
2) screening for drugs that act on the target genes measured to be highly expressed in step 1).

In the method of the present invention, the biological sample in step 1) includes a variety of biological samples, specifically blood, serum, plasma, tissue, cell, lymph, bone marrow fluid, saliva, urine, feces, ocular fluid, semen, brain extracts, cerebrospinal fluid, synovial fluid, pleural effusion, ascitic fluid, or amniotic fluid, and more specifically, it is a tissue or cell, and even more specifically, it is cancer tissue or cancer cells.

In the method of the present invention, the target genes must include ADA and CD52 in step 1), and may include at least 10, 15, 20, 21, 22, 23, 24, 25, 26, 27, 28, 29, 30, 31, 32, 33, 34, 35, 36, 37, 38, 39, 40, 41, 42, 43, 44, or 45 target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ, or may include all of the above targets, but is not limited thereto.

Additionally, in step 1) of the method of the present invention, at least 10 target genes may be further included, which are selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB, more specifically including at least 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 target genes, or may include all of the above target genes, but is not limited thereto.

In the method of the present invention, measuring the expression of the genes in step 1) may be performed according to a digital PCR method, specifically being performed using a droplet digital PCR method.

In the method of the present invention, the pharmaceutical acting on the target genes in step 2) may be an anticancer agent that has been approved by the U.S. FDA or the European EMA for clinical use, but is not limited thereto. For example, anticancer agents that have been approved by the U.S. FDA for clinical use are described in The Author(s) BMC Systems Biology 2017, 11 (Suppl 5): 87.

More specifically, the method for screening personalized therapeutic agents for cancer patients using the droplet digital PCR method may be performed through the following steps:
a) isolating cancer tissue and normal tissue from a cancer patient;
b) isolating total RNA from the isolated cancer tissue and normal tissue, and synthesizing cDNA using the total RNA as a template;
c) using the synthesized cDNA as a template, generating a plurality of droplets from a reaction mixture comprising primers or probes that specifically hybridize to the target genes ADA and CD52, and primers or probes that specifically hybridize to at least 5 target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ;
d) amplifying the target nucleic acid sequences in the generated droplets;
e) determining the amount of amplified target nucleic acids by counting the number of droplets containing the amplified target nucleic acids, and comparing the amounts of amplified target nucleic acids in normal tissue and cancer tissue to select the target genes that are highly expressed in cancer tissue compared to normal tissue; and
f) screening therapeutic agents that act on the selected target genes.

In the present invention, the expression changes of 134 cancer therapeutic target genes between the normal and cancer tissues of cancer patients were quantified using digital PCR, revealing differences in the expression patterns of cancer therapeutic targets among individual cancer patients. Furthermore, it was confirmed that digital PCR enables more accurate analysis compared to qPCR. Therefore, measurement of the expression patterns of the 134 cancer therapeutic target genes through digital PCR according to the present invention can be utilized for the selection of personalized cancer therapies for cancer patients.

Furthermore, the present invention provides a method for screening personalized therapeutic agents for cancer patients, comprising the following steps:
1) isolating and proliferating cancer cells and normal cells from cancer tissue and normal tissue obtained from a cancer patient;
2) treating the cancer cells from step 1) with a candidate anticancer agent;
3) isolating total RNA from the cancer cells of step 1) and normal cells of step 1), and from the cancer cells treated with the candidate anticancer agent of step 2), and synthesizing cDNA using the total RNA as a template;
4) using the synthesized cDNA of step 3) as a template, generating multiple droplets from a reaction solution containing primers or probes that specifically hybridize to the target genes ADA and CD52, as well as primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ;
5) amplifying the target nucleic acid sequences in the droplets generated in step 4); and
6) counting the number of droplets containing the amplified target nucleic acids from step 5) to determine the amount of amplified target nucleic acid, followed by comparing the amount of amplified target nucleic acid in normal cells, cancer cells, and cancer cells treated with the candidate anticancer agent.

In the method of the present invention, the method for isolating cancer cells and normal cells from the cancer tissue and normal tissue obtained from a cancer patient in step 1) may utilize any method known in the art. For example, the cancer tissue or normal tissue may be digested using tissue dissociation enzymes or mechanical methods, and the dissociated cancer cells may be separated based on cell size, density, or surface characteristics using techniques such as density gradient centrifugation, cell sorters, or magnetic separation methods.

In the method of the present invention, the candidate anticancer agent in step 2) may include any substance, molecule, element, compound, entity, or a combination thereof. For example, but not limited to, it may include a protein, polypeptide, small organic molecule, polysaccharide, polynucleotide, and the like. Additionally, it may comprise a natural product, synthetic compound, or chemical compound, or a combination of two or more substances. A specific example includes a polypeptide, beta-turn mimetics, polysaccharide, phospholipid, hormone, prostaglandin, steroid, aromatic compound, heterocyclic compound, benzodiazepine, oligomeric N-substituted glycine, oligocarbamates, saccharides, fatty acid, purine, pyrimidine, or its derivative, structural analog, or combination thereof, and may be a synthetic substance, while another candidate anticancer agent may be a natural product.

In the method of the present invention, the method for isolating total RNA in step 3) may utilize any method known in the art, and, for example, the phenol extraction method may be employed.

In the method of the present invention, in step 4), ADA and CD52 must be included, and at least 10, 15, 20, 21, 22, 23, 24, 25, 26, 27, 28, 29, 30, 31, 32, 33, 34, 35, 36, 37, 38, 39, 40, 41, 42, 43, 44, or 45 target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ may further be included, or all of the listed target genes may be included, but is not limited thereto.

Additionally, in step 4), at least 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB may be included, or all of the listed target genes may be included, but are not limited thereto.

In step 4), the primers or probes may be primers or probes for digital PCR, specifically primers or probes for droplet digital PCR. The droplet digital PCR is a system that partitions the PCR reaction mixture into approximately 15,000 to 20,000 droplets, where amplification is carried out and the target nucleic acid is subsequently counted. Depending on whether amplification of the target nucleic acid occurs in each droplet, the droplets are counted as positive droplets (1) or negative droplets (0), similar to a digital signal. The copy number of the target nucleic acid is calculated using Poisson distribution, and the result is ultimately determined as the copy number per sample volume (μl).

Additionally, the reaction mixture may include probes such as FAM, HEX, or VIC, or a fluorescent dye like EvaGreen, which is dispensed along with oil into a cartridge and set in a droplet generator to produce approximately 20,000 droplets.

In the method of the present invention, in step 5), amplification of the target nucleic acid sequence may be carried out through multiplex PCR. Multiplex PCR refers to a method of performing PCR amplification in a single reaction tube using multiple sets of primers simultaneously. For example, by performing multiplex PCR with primer sets for the target genes in a single reaction tube, the gene expression levels can be determined simultaneously.

In the method of the present invention, in step 6), the PCR plate with completed PCR may be mounted on a droplet reader for digital PCR to check and count the EvaGreen fluorescence values of the droplets through QuantaSoft, thereby enabling the analysis of the final copy number of the amplified target nucleic acid; however, this is not limited thereto.

Ideally, the label is a fluorescent label, and the fluorescent labeling substance may be FAM (5- or 6-carboxyfluorescein), VIC, NED, fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor, PET, Biosearch Blue™, Marina Blue, Bothell Blue, 350 FAM™, SYBR Green I, fluorescein, EvaGreen™, Alexa Fluor 488 JOE™, VIC™, HEX™, TET™, CAL Fluor Gold 540, Yakima Yellow, ROX™, CAL Fluor Red 610, Cy3.5™, Texas Red, 568 Cy5™, Quasar™ 670, LightCycler Red640, Alexa Fluor 633, Quasar™ 705, LightCycler Red705, Alexa Fluor 680, Cyto 9, LC Green, or LC Green Plus+, and specifically, it may be FAM, HEX, SYBR Green, or EvaGreen; however, it is not limited thereto.

The fluorescent labeling substances have different excitation and emission wavelengths depending on their types, and their methods of use also vary. Therefore, when selecting fluorescent labeling substances for use together in a single PCR reaction, it is necessary to determine whether they can be detected separately and to select them accordingly, allowing for the use of different colors. Specific details and selection of the fluorescent labeling substances are evident to those skilled in the art pertaining to the present invention.

In the method of the present invention, in step 6), the amount of the amplified target nucleic acid increases in cancer cells compared to normal cells, and decreases in cancer cells treated with candidate anticancer agents compared to untreated cancer cells, thereby allowing the selection of candidate agents as personalized medicine for cancer patients.

In the present invention, the expression changes of 134 cancer therapeutic target genes between the normal and cancer tissues of cancer patients were quantified using digital PCR, revealing differences in the expression patterns of cancer therapeutic targets among individual cancer patients. Furthermore, it was confirmed that digital PCR enables more accurate analysis compared to qPCR. Therefore, by measuring the expression patterns of the 134 cancer therapeutic target genes through digital PCR according to the present invention, it is possible to screen suitable medicines for cancer patients, which can be utilized for the screening of a personalized agent for individual cancer patients.

Hereinafter, the present invention will be described in detail with reference to specific examples.

However, the following examples are merely illustrative of the present invention and do not limit the scope of the invention in any way.

### <Example 1> Preparation of Sample

Samples of normal tissue and cancer tissue were obtained from three renal cancer patients from the Pathology Department specimen bank at Wonju Severance Christian Hospital. Three pairs of normal and cancer tissue samples were stored in cryo-tubes at −70° C. until RNA extraction. Total RNA extraction was carried out according to the manufacturer's protocol using the commercially available RNeasy Midi Kit (Qiagen, Chatsworth, CA, USA). The quantity and quality of the extracted total RNA were assessed using ultraviolet spectrophotometry (DU 530, Beckman, USA).

Two micrograms of the extracted total RNA were subjected to reverse transcription reactions in a volume of 20 μl under the conditions of 2 units of DNase I (4.2 μM MgCl₂). The reaction mixture was prepared with the following composition: 2 μg of total RNA, 3.68 μl of 50 mM MgCl₂, 0.96 μl of DNase I, and the volume was adjusted to 20 μl with DEPC-water.

For the samples treated with DNase I, reverse transcription reactions were performed using the Superscript II Reverse Transcription Kit (Invitrogen cat #18064-071) under the following conditions: 20 μl of 5× First Strand buffer, 10 μl of 100 mM DTT, 20 μl of 10 mM dNTPs, 5 μl of pdN6 (1.6 μg/μl), 0.5 μl of RTase (200 U/μl), 20 μl of RNA, and 24.5 μl of DEPC-water. The total volume of the reaction mixture was adjusted to 100 μl.

The reverse transcription reaction was conducted under the following temperature conditions: 25° C. for 10 minutes, 42° C. for 50 minutes, 72° C. for 10 minutes, followed by a hold at 4° C.

After the completion of the reverse transcription reaction, the mixture was adjusted to a concentration of 5 ng/μl of cDNA using DEPC-water.

### <Example 2> Digital PCR Analysis for the Selection of Personalized Agents for Cancer Patients

For the selection of personalized agents for cancer patients, the cDNA obtained in Example 1 was used as a template, and primers specific to the 48 target genes listed in [Table 1] were employed to complete the mRNA expression profile of the 48 targets using the droplet digital PCR (ddPCR) method.

Additionally, primers specific to the 86 target genes listed in [Table 2] were further utilized to complete the mRNA expression profile of the 134 targets using the ddPCR method.

Specifically, among approximately 300 cancer therapeutics approved by the U.S. FDA or the European EMA, including renal cancer therapeutics such as the mTOR inhibitor everolimus, a total of 48 and 86 suitable molecular biological targets were selected for the present invention. The ddPCR primers for these targets were designed as shown in [Table 1] and [Table 2].

TABLE 1

| | | | GenBank | |
|---|---|---|---|---|
| | Gene | Disclosure | Accession | |
| Number | name | Status | Number | Primer Sequence(5′→3′) |
| 1 | ABL1 | No | NM_005157 | (Forward) |
| | | | | TAACACTCTAAGCATAACTAA |
| | | | | AGGT (Sequence Number 1) |
| | | | | (Reverse) |
| | | | | CACACCATTCCCCATTGTGAT |
| | | | | T (Sequence Number 2) |
| 2 | ABL2 | No | NM_007314 | (Forward) |
| | | | | TGACCCTAATCTCTTCGTTGC |
| | | | | A (Sequence Number 3) |
| | | | | (Reverse) |
| | | | | TGTAACCAAGGACTCGTAGCT |
| | | | | TTT (Sequence Number 4) |
| 3 | ALAD | No | NM_001003945 | (Forward) |
| | | | | TGAGGCCCTTGGTGGA |
| | | | | (Sequence Number 5) |
| | | | | (Reverse) |
| | | | | CGCTCGTCCTTGGGAACT |
| | | | | (Sequence Number 6) |
| 4 | EGFR | No | NM_005228 | (Forward) |
| | | | | TGCCCGCATTAGCTCTTAGA |
| | | | | (Sequence Number 7) |
| | | | | (Reverse) |
| | | | | GTGCCCGAGGTGGAAGTACT |
| | | | | (Sequence Number 8) |
| 5 | B-RAF | No | NM_004333 | (Forward) |
| | | | | CAACAACAGGGACCAGATAA |
| | | | | TTTT (Sequence Number 9) |
| | | | | (Reverse) |
| | | | | CGTACCTTACTGAGATCTGGA |
| | | | | GACA (Sequence Number 10) |
| 6 | CD20 | No | NM_152866 | (Forward) |
| | | | | TGTCTTCACTGGTGGGCC |
| | | | | (Sequence Number 11) |
| | | | | (Reverse) |
| | | | | AGCCCATTCATAATCTGGACA |
| | | | | (Sequence Number 12) |
| 7 | CD33 | No | NM_001772 | (Forward) |
| | | | | CGCTCTTTGTCTCTGCCTCAT |
| | | | | (Sequence Number 13) |
| | | | | (Reverse) |
| | | | | CTGCTGTCCTGGCTGCTTT |
| | | | | (Sequence Number 14) |
| 8 | CHD1 | No | NM_001364113 | (Forward) |
| | | | | CTTCAGGAACAGAACGAACA |
| | | | | GG (Sequence Number 15) |
| | | | | (Reverse) |
| | | | | GGAGATGACTAGTTTGGCATT |
| | | | | CA (Sequence Number 16) |
| 9 | C-RAF | No | NM_001354689 | (Forward) |
| | | | | GGTAGCTGACTGTGTGAAGA |
| | | | | (Sequence Number 17) |
| | | | | (Reverse) |
| | | | | CAGTGTTGGAGCAGCTCAAT |
| | | | | (Sequence Number 18) |
| 10 | CSF1R | No | NM_005211 | (Forward) |
| | | | | AGCAGGCCCAAGAGGAC |
| | | | | (Sequence Number 19) |
| | | | | (Reverse) |
| | | | | CCGCTGCCACCGCTTC |
| | | | | (Sequence Number 20) |
| 11 | DHFR | No | NM_000791 | (Forward) |
| | | | | CTGCATCGTCGCTGTGTCC |
| | | | | (Sequence Number 21) |
| | | | | (Reverse) |
| | | | | GTTGTGGTCATTCTCTGGAAA |
| | | | | TAT (Sequence Number 22) |
| 12 | DNMT1 | No | NM_001130823 | (Forward) |
| | | | | GCACCTCATTTGCCGAATAC |
| | | | | (Sequence Number 23) |
| | | | | (Reverse) |
| | | | | CTCCTGCATCAGCCCAAATA |
| | | | | (Sequence Number 24) |
| 13 | EPHA2 | No | NM_004431 | (Forward) |
| | | | | GTCCGAGTGGCTGGAGT |
| | | | | (Sequence Number 25) |
| | | | | (Reverse) |
| | | | | ACCACCTTCTCGATGGCA |
| | | | | (Sequence Number 26) |
| 14 | FLT1 | No | NM_002019 | (Forward) |
| | | | | TGCGAGCTCCGGCTTT |
| | | | | (Sequence Number 27) |
| | | | | (Reverse) |
| | | | | CCTTGTAGAAACCGTCAGAAT |
| | | | | C (Sequence Number 28) |
| 15 | FLT3 | No | NM_004119 | (Forward) |
| | | | | ACCCCACTTTCCAATCACATC |
| | | | | (Sequence Number 29) |
| | | | | (Reverse) |
| | | | | CGAGTCCGGGTGTATCTGAA |
| | | | | (Sequence Number 30) |
| 16 | FLT4 | No | NM_182925 | (Forward) |
| | | | | TCCTGGCTTCCCGAAAGT |
| | | | | (Sequence Number 31) |
| | | | | (Reverse) |
| | | | | GGCCAAAGTCACAGATCTTCA |
| | | | | C (Sequence Number 32) |
| 17 | FYN | No | NM_002037 | (Forward) |
| | | | | ATCCGCGAGAGTGAAACCA |
| | | | | (Sequence Number 33) |
| | | | | (Reverse) |
| | | | | TCATCCCAATCACGGATAGAA |
| | | | | (Sequence Number 34) |
| 18 | GARFT | No | NM_000819 | (Forward) |
| | | | | GCAGCCCGAGTACTTATAATT |
| | | | | GG (Sequence Number 35) |
| | | | | (Reverse) |
| | | | | GATGAGACTGTGCAAGTTTCC |
| | | | | A (Sequence Number 36) |
| 19 | HDAC1 | No | NM_004964 | (Forward) |
| | | | | TCCGCATGACTCATAATTTGC |
| | | | | (Sequence Number 37) |
| | | | | (Reverse) |
| | | | | TGAGGGCGATAGATTTCCATT |
| | | | | (Sequence Number 38) |
| 20 | HDAC6 | No | NM_001321225 | (Forward) |
| | | | | GGAATGGCATGGCCATCATTA |
| | | | | G (Sequence Number 39) |
| | | | | (Reverse) |
| | | | | CGTGGTTGAACATGCAATAGC |
| | | | | (Sequence Number 40) |
| 21 | HER2 | No | NM_004448 | (Forward) |
| | | | | CGGCAGCAGAAGATCCGG |
| | | | | (Sequence Number 41) |
| | | | | (Reverse) |
| | | | | CGCTAGGTGTCAGCGGCT |
| | | | | (Sequence Number 42) |
| 22 | KDR | No | NM_002253 | (Forward) |
| | | | | TTCTTGGCATCGCGAAAGTG |
| | | | | (Sequence Number 43) |
| | | | | (Reverse) |
| | | | | ATTTTAACCACGTTCTTCTCC |
| | | | | GATA (Sequence Number 44) |
| 23 | Kit | No | NM_000222 | (Forward) |
| | | | | CAACCAAGGCCGACAAAA |
| | | | | (Sequence Number 45) |
| | | | | (Reverse) |
| | | | | TGATGGCGGGAGTCACAT |
| | | | | (Sequence Number 46) |
| 24 | Lck | No | NM_001042771 | (Forward) |
| | | | | TCCTGCTGACGGAAATTGTC |
| | | | | (Sequence Number 47) |
| | | | | (Reverse) |
| | | | | ACCTCCGGGTTGGTCATC |
| | | | | (Sequence Number 48) |
| 25 | mTOR | No | NM_004958 | (Forward) |
| | | | | CCGAGCGACGAGAGATCAT |
| | | | | (Sequence Number 49) |
| | | | | (Reverse) |
| | | | | CAAGGGACCGCACCATAAG |
| | | | | (Sequence Number 50) |
| 26 | NK1R | No | NM_015727 | (Forward) |
| | | | | CAACGAATGGTACTACGGCC |
| | | | | (Sequence Number 51) |
| | | | | (Reverse) |
| | | | | GGATGTATGATGGCCATGTCA |
| | | | | (Sequence Number 52) |
| 27 | PDGFRα | No | NM_006206 | (Forward) |
| | | | | TGCCCGAGGAATGGAGTT |
| | | | | (Sequence Number 53) |
| | | | | (Reverse) |
| | | | | CTTGTGCCAGGAGGACGTT |
| | | | | (Sequence Number 54) |
| 28 | PDGFRβ | No | NM_002609 | (Forward) |
| | | | | GCGCTGGCGAAATCG |
| | | | | (Sequence Number 55) |
| | | | | (Reverse) |
| | | | | TTCACGCGAACCAGTGTCA |
| | | | | (Sequence Number 56) |
| 29 | PolA | No | NM_001330360 | (Forward) |
| | | | | AGTGAAACAAGAGGCGGATT |
| | | | | C (Sequence Number 57) |
| | | | | (Reverse) |
| | | | | CAAGAGACATCCGGGAGAAA |
| | | | | A (Sequence Number 58) |
| 30 | PolB | No | NM_002690 | (Forward) |
| | | | | GAATCACCGACATGCTCACA |
| | | | | (Sequence Number 59) |
| | | | | (Reverse) |
| | | | | GGATAGCTTGGCTCACGTTCT |
| | | | | (Sequence Number 60) |
| 31 | PSMB5 | No | NM_002797 | (Forward) |
| | | | | CAGAAGAGCCAGGAATCGAA |
| | | | | (Sequence Number 61) |
| | | | | (Reverse) |
| | | | | CAACTATGACTCCATGGCGGA |
| | | | | (Sequence Number 62) |
| 32 | Ret | No | NM_001406743 | (Forward) |
| | | | | TGGAGACCCAAGACATCAAC |
| | | | | AT (Sequence Number 63) |
| | | | | (Reverse) |
| | | | | TCCCCCAACAATGCTGC |
| | | | | (Sequence Number 64) |
| 33 | RRM1 | No | NM_001033 | (Forward) |
| | | | | TGCACTTCTACGGCTGGAA |
| | | | | (Sequence Number 65) |
| | | | | (Reverse) |
| | | | | AGCCGCTGGTCTTGTCCTTA |
| | | | | (Sequence Number 66) |
| 34 | RRM2 | No | NM_001165931 | (Forward) |
| | | | | TCTGGCTTTCTTTGCAGCAA |
| | | | | (Sequence Number 67) |
| | | | | (Reverse) |
| | | | | CTTCTTGGCTAAATCGCTCCA |
| | | | | (Sequence Number 68) |
| 35 | RRM2B | No | NM_015713 | (Forward) |
| | | | | AGGCACAGGCTTCCTTCTG |
| | | | | (Sequence Number 69) |
| | | | | (Reverse) |
| | | | | GCTTGTTCCAGTGAGGGAGAT |
| | | | | (Sequence Number 70) |
| 36 | SRC | No | NM_005417 | (Forward) |
| | | | | TTCAGAGGAGCCCATTTACAT |
| | | | | C (Sequence Number 71) |
| | | | | (Reverse) |
| | | | | CCTTGAGAAAGTCCAGCAAA |
| | | | | CT (Sequence Number 72) |
| 37 | TLR7 | No | NM_016562 | (Forward) |
| | | | | ACCAGACCTCTACATTCCATT |
| | | | | T (Sequence Number 73) |
| | | | | (Reverse) |
| | | | | GATAAGAATTTGTCTCTTCAG |
| | | | | TGT (Sequence Number 74) |
| 38 | TOP1 | No | NM_003286 | (Forward) |
| | | | | GCTAAAAGAACTGACAGCCC |
| | | | | (Sequence Number 75) |
| | | | | (Reverse) |
| | | | | AAGAATTGCAACAGCTCGATT |
| | | | | G (Sequence Number 76) |
| 39 | TOP2A | No | NM_001067 | (Forward) |
| | | | | CAGTGAAGAAGACAGCAGCA |
| | | | | AAAA (Sequence Number 77) |
| | | | | (Reverse) |
| | | | | AGCTGGATCCCTTTTAGTTCC |
| | | | | TT (Sequence Number 78) |
| 40 | TYMS | No | NM_001071 | (Forward) |
| | | | | ATCACATCGAGCCACTGAAAA |
| | | | | (Sequence Number 79) |
| | | | | (Reverse) |
| | | | | TCCTGAGCTTTGTGAAAGGT |
| | | | | (Sequence Number 80) |
| 41 | YES | No | NM_005433 | (Forward) |
| | | | | GCAGACTTTGGTTTAGCAAGG |
| | | | | T (Sequence Number 81) |
| | | | | (Reverse) |
| | | | | ACAGTGCAGCTTCAGGAGC |
| | | | | (Sequence Number 82) |
| 42 | CYP19A1 | No | NM_000103 | (Forward) |
| | | | | GGATCCCTTTGGACGAAAGT |
| | | | | (Sequence Number 83) |
| | | | | (Reverse) |
| | | | | AGCTTGCCATGCATCAAAAT |
| | | | | (Sequence Number 84) |
| 43 | AR | No | NM_000044 | (Forward) |
| | | | | GAATTCCTGTGCATGAAAGCA |
| | | | | (Sequence Number 85) |
| | | | | (Reverse) |
| | | | | TTGATGTAGTTCATTCGAAGT |
| | | | | TCA (Sequence Number 86) |
| 44 | ERα | No | NM_000125 | (Forward) |
| | | | | CCTGTGAGGGCTGCAAG |
| | | | | (Sequence Number 87) |
| | | | | (Reverse) |
| | | | | TCTTCCTCCTGTTTTTATCAAT |
| | | | | GG (Sequence Number 88) |
| 45 | ERβ | No | NM_001291723 | (Forward) |
| | | | | AAGTTGGCCGACAAGGAGTT |
| | | | | (Sequence Number 89) |
| | | | | (Reverse) |
| | | | | ACAGGCTGAGCTCCACAAAG |
| | | | | (Sequence Number 90) |
| 46 | RXRα | No | NM_002957 | (Forward) |
| | | | | GAGCCCAAGACCGAGACCTA |
| | | | | (Sequence Number 91) |
| | | | | (Reverse) |
| | | | | AGCTGTTTGTCGGCTGCTT |
| | | | | (Sequence Number 92) |
| 47 | RXRβ | No | NM_001270401 | (Forward) |
| | | | | AGCCCCCAGATTAACTCAACA |
| | | | | (Sequence Number 93) |
| | | | | (Reverse) |
| | | | | GATTGCACATAGCCGTTTGC |
| | | | | (Sequence Number 94) |
| 48 | RXRγ | No | NM_006917 | (Forward) |
| | | | | GAAGTTTCCCGCAGGCTATG |
| | | | | (Sequence Number 95) |
| | | | | (Reverse) |
| | | | | TGATGGGCTCATGGATGTAGA |
| | | | | (Sequence Number 96) |

TABLE 2

| | | | GenBank | |
|---|---|---|---|---|
| | Gene | Disclosure | Accession | |
| Number | name | Status | Number | Primer Sequence(5′→ 3') |
| 49 | ACPP | No | NM_001099 | (Forward) |
| | | | | TTGTACTTTGAGAAGGGGGA |
| | | | | GT (Sequence Number 97) |
| | | | | (Reverse) |
| | | | | GGTAGCATGAGGGGATACGG |
| | | | | (Sequence Number 98) |
| 50 | ADA | No | NM_000022 | (Forward) |
| | | | | AAGAGGAGTTTAAAAGGCTG |
| | | | | AACA (Sequence Number 99) |
| | | | | (Reverse) |
| | | | | AAGGTGGCATCCCATAGGC |
| | | | | (Sequence Number 100) |
| 51 | ALK | No | NM_004304 | (Forward) |
| | | | | ATTGGAGACTTCGGGATGGC |
| | | | | (Sequence Number 101) |
| | | | | (Reverse) |
| | | | | CTTAACTGGCAGCATGGCAC |
| | | | | (Sequence Number 102) |
| 52 | BCR- | No | NM_005157 | (Forward) |
| | ABL-1 | | | GGCCAGTGGAGATAACACTCT |
| | | | | AA (Sequence Number 103) |
| | | | | (Reverse) |
| | | | | CACCATTCCCCATTGTGATTAT |
| | | | | A (Sequence Number 104) |
| 53 | BTK | No | NM_000061 | (Forward) |
| | | | | AGAAGCACCTTTTCAGCACC |
| | | | | A (Sequence Number 105) |
| | | | | (Reverse) |
| | | | | ACACTGGATATTTGAGCCTGG |
| | | | | AT (Sequence Number 106) |
| 54 | CD19 | No | NM_001178098 | (Forward) |
| | | | | ATGAAGAGCTGACCCAGCCG |
| | | | | (Sequence Number 107) |
| | | | | (Reverse) |
| | | | | CCTCATAGGACTGGGACCCT |
| | | | | (Sequence Number 108) |
| 55 | CD3D | No | NM_000732 | (Forward) |
| | | | | GAACATAGCACGTTTCTCTCT |
| | | | | GG (Sequence Number 109) |
| | | | | (Reverse) |
| | | | | GTATCTTGAAGGGGCTCACTT |
| | | | | G (Sequence Number 110) |
| 56 | CD52 | No | NM_001803 | (Forward) |
| | | | | TTCCTCTTCCTCCTACTCACC |
| | | | | A (Sequence Number 111) |
| | | | | (Reverse) |
| | | | | CTGGTGTCGTTTTGTCCTGA |
| | | | | (Sequence Number 112) |
| 57 | C-MET | No | NM_001127500 | (Forward) |
| | | | | GGACATCAGAGGGTCGCTT |
| | | | | (Sequence Number 113) |
| | | | | (Reverse) |
| | | | | GATGGGAGTCCAGGAGAAAA |
| | | | | T (Sequence Number 114) |
| 58 | CRBN | No | NM_016302 | (Forward) |
| | | | | GACGCTGCGCACAACAT |
| | | | | (Sequence Number 115) |
| | | | | (Reverse) |
| | | | | CTGGTCTTCAACTTCCATTTC |
| | | | | ATC (Sequence Number 116) |
| 59 | CTLA4 | No | NM_005214 | (Forward) |
| | | | | GTACCCACCGCCATACTACC |
| | | | | (Sequence Number 117) |
| | | | | (Reverse) |
| | | | | GCATTTTCACATAGACCCCTG |
| | | | | (Sequence Number 118) |
| 60 | CYP17A1 | No | NM_000102 | (Forward) |
| | | | | GTTCAAGGATGGCGATCAG |
| | | | | (Sequence Number 119) |
| | | | | (Reverse) |
| | | | | GGCCAGCATATCACACAATGT |
| | | | | (Sequence Number 120) |
| 61 | DDR2 | No | NM_001014796 | (Forward) |
| | | | | CCCAGGAAACTCCTAACTTTC |
| | | | | AA (Sequence Number 121) |
| | | | | (Reverse) |
| | | | | TCCCTCCACTTCACAGAGATG |
| | | | | (Sequence Number 122) |
| 62 | ERBB4 | No | NM_005235 | (Forward) |
| | | | | GAACAGCAGTACCGAGCCTT |
| | | | | (Sequence Number 123) |
| | | | | (Reverse) |
| | | | | ACTTCTCGAACAGACCGCAG |
| | | | | (Sequence Number 124) |
| 63 | FCGR1A | No | NM_00137880 | (Forward) |
| | | | | TCCTTAAGCGCAGCCCT |
| | | | | (Sequence Number 125) |
| | | | | (Reverse) |
| | | | | AGGACATGAAACCAGACAGG |
| | | | | A (Sequence Number 126) |
| 64 | FGF1 | No | NM_000800 | (Forward) |
| | | | | GGGCTTTTATACGGCTCACAG |
| | | | | AC (Sequence Number 127) |
| | | | | (Reverse) |
| | | | | AATGGTTCTCCTCCAGCCTT |
| | | | | (Sequence Number 128) |
| 65 | FGFR1 | No | NM_023110 | (Forward) |
| | | | | GACGCAGGGGAGTATACGTG |
| | | | | (Sequence Number 129) |
| | | | | (Reverse) |
| | | | | TCTCTTCCAGGGCTTCCAGA |
| | | | | (Sequence Number 130) |
| 66 | FGFR2 | No | NM_000141 | (Forward) |
| | | | | CACGACCAAGAAGCCAGACT |
| | | | | (Sequence Number 131) |
| | | | | (Reverse) |
| | | | | TGGACTCAGCCGAAACTGTTA |
| | | | | (Sequence Number 132) |
| 67 | FGFR3 | No | NM_000142 | (Forward) |
| | | | | GAATGCCTCCCACGAGGAC |
| | | | | (Sequence Number 133) |
| | | | | (Reverse) |
| | | | | GAGGATGGAGCGTCTGTCAC |
| | | | | (Sequence Number 134) |
| 68 | FRK | No | NM_002031 | (Forward) |
| | | | | CCGATGTATGGTCATTTGGAA |
| | | | | T (Sequence Number 135) |
| | | | | (Reverse) |
| | | | | CTGGATTACCTGGGCACCT |
| | | | | (Sequence Number 136) |
| 69 | GNRH1 | No | NM_000825 | (Forward) |
| | | | | GGTTGGTCAACTGGCAGAAA |
| | | | | C (Sequence Number 137) |
| | | | | (Reverse) |
| | | | | AGAGCTCCTTTCAGGTCTCG |
| | | | | (Sequence Number 138) |
| 70 | GNRHR | No | NM_000406 | (Forward) |
| | | | | ATCTTCACCCTGACACGGG |
| | | | | (Sequence Number 139) |
| | | | | (Reverse) |
| | | | | TTAGAGTCTTCAGCCGTGCTC |
| | | | | (Sequence Number 140) |
| 71 | HDAC2 | No | NM_001527 | (Forward) |
| | | | | TCTGCTACTACTACGACGGTG |
| | | | | AT (Sequence Number 141) |
| | | | | (Reverse) |
| | | | | CAGTGGCTTTATGGGGCCTA |
| | | | | (Sequence Number 142) |
| 72 | HDAC3 | No | NM_001355039 | (Forward) |
| | | | | TGGCATTGATGACCAGAGTTA |
| | | | | CA (Sequence Number 143) |
| | | | | (Reverse) |
| | | | | TGCACGTGGGTTGGTAGAAG |
| | | | | (Sequence Number 144) |
| 73 | HPRT1 | No | NM_000194 | (Forward) |
| | | | | CTGGCGTCGTGATTAGTGAT |
| | | | | (Sequence Number 145) |
| | | | | (Reverse) |
| | | | | GAGCAAGACGTTCAGTCCTG |
| | | | | T (Sequence Number 146) |
| 74 | IFNAR1 | No | NM_001384498 | (Forward) |
| | | | | TGCTGCGAAAGTCTTCTTGAG |
| | | | | (Sequence Number 147) |
| | | | | (Reverse) |
| | | | | TGGCTGTTCAGAGAAATACTCATC |
| | | | | (Sequence Number 148) |
| 75 | IFNAR2 | No | NM_207585 | (Forward) |
| | | | | TGAGCCAGAATGCCTTCATC |
| | | | | (Sequence Number 149) |
| | | | | (Reverse) |
| | | | | AGATTCATCTGTGTAATCAGG |
| | | | | CG (Sequence Number 150) |
| 76 | IL2RA | No | NM_000417 | (Forward) |
| | | | | GAATGCAAGAGAGGTTTCCGC |
| | | | | (Sequence Number 151) |
| | | | | (Reverse) |
| | | | | TGTTCCGAGTGGCAGAGCTT |
| | | | | (Sequence Number 152) |
| 77 | IL2RB | No | NM_000878 | (Forward) |
| | | | | ACACTTCCTGCCAAGTCCA |
| | | | | (Sequence Number 153) |
| | | | | (Reverse) |
| | | | | ATGCTTGACTCACGGGGA |
| | | | | (Sequence Number 154) |
| 78 | IL2RG | No | NM_000206 | (Forward) |
| | | | | ATTTCTGGCTGGAACGGACG |
| | | | | (Sequence Number 155) |
| | | | | (Reverse) |
| | | | | GCCAGTCCCTTAGACACACC |
| | | | | (Sequence Number 156) |
| 79 | ITK | No | NM_005546 | (Forward) |
| | | | | CCTCCTACTCCTGAAGACAAC |
| | | | | AG (Sequence Number 157) |
| | | | | (Reverse) |
| | | | | TGAGGATCATTGGTTTGGTAG |
| | | | | TC (Sequence Number 158) |
| 80 | JAK1 | No | NM_002227 | (Forward) |
| | | | | CACAGGCATGCCGTATCTCT |
| | | | | (Sequence Number 159) |
| | | | | (Reverse) |
| | | | | CCAGAGCTTGGTGTTCTCGT |
| | | | | (Sequence Number 160) |
| 81 | JAK2 | No | NM_004972 | (Forward) |
| | | | | TGCTGCTTCTAAAGCTTGTGG |
| | | | | TAT (Sequence Number 161) |
| | | | | (Reverse) |
| | | | | GGAAGACATGGTTGGGTGGA |
| | | | | T (Sequence Number 162) |
| 82 | LDLR | No | NM_000527 | (Forward) |
| | | | | TGACAATGTCTCACCAAGCTC |
| | | | | T(Sequence Number 163) |
| | | | | (Reverse) |
| | | | | GAGGACAATGGACAGAGCCC |
| | | | | (Sequence Number 164) |
| 83 | LHCGR | No | NM_000233 | (Forward) |
| | | | | CCATCTCAAGCTTTCAGAGGA |
| | | | | (Sequence Number 165) |
| | | | | (Reverse) |
| | | | | TAGCTTCTATCCTTTCCAGGG |
| | | | | A (Sequence Number 166) |
| 84 | LIMK1 | No | NM_002314 | (Forward) |
| | | | | TGACACACCGTGAGACAGGT |
| | | | | (Sequence Number 167) |
| | | | | (Reverse) |
| | | | | GCATGACCTTCACCTCCTTGA |
| | | | | (Sequence Number 168) |
| 85 | MAP1A | No | NM_002373 | (Forward) |
| | | | | GGGAGAATCTTCAGGTGACTC |
| | | | | (Sequence Number 169) |
| | | | | (Reverse) |
| | | | | CCAGGACCAGGACATTCAGT |
| | | | | (Sequence Number 170) |
| 86 | MAP2 | No | NM_002374 | (Forward) |
| | | | | CCATCACCTGCCTCAGAAC |
| | | | | (Sequence Number 171) |
| | | | | (Reverse) |
| | | | | AATACACTGGGAGCCAGAGC |
| | | | | (Sequence Number 172) |
| 87 | MAP2K1 | No | NM_002755 | (Forward) |
| | | | | AGCTCTGCGGAGACCAACT |
| | | | | (Sequence Number 173) |
| | | | | (Reverse) |
| | | | | GCTGCTGCTCATCAAGCTCT |
| | | | | (Sequence Number 174) |
| 88 | MAP2K2 | No | NM_030662 | (Forward) |
| | | | | TATATTGTGAACGAGCCACCT |
| | | | | CC (Sequence Number 175) |
| | | | | (Reverse) |
| | | | | TGGTTTGTGAGCATCTTCAGG |
| | | | | T (Sequence Number 176) |
| 89 | MAPK11 | No | NM_002751 | (Forward) |
| | | | | CGAGGACTTCAGCGAAGTGT |
| | | | | A (Sequence Number 177) |
| | | | | (Reverse) |
| | | | | GCCGAGTGGATGTACTTCAG |
| | | | | (Sequence Number 178) |
| 90 | MET | No | NM_001127500 | (Forward) |
| | | | | GAGCACTGCTTTAATAGGACA |
| | | | | C (Sequence Number 179) |
| | | | | (Reverse) |
| | | | | GTTCGATATTCATCACGGCG |
| | | | | (Sequence Number 180) |
| 91 | NEK11 | No | NM_024800 | (Forward) |
| | | | | CCAAACGAGGAGAGGAATTA |
| | | | | AAG (Sequence Number 181) |
| | | | | (Reverse) |
| | | | | GCCTGTACAGTTTCATTTGGA |
| | | | | TTT (Sequence Number 182) |
| 92 | NR3C1 | No | NM_000176 | (Forward) |
| | | | | AAGTTTTCTTCAAAAGAGCA |
| | | | | GTGG (Sequence Number 183) |
| | | | | (Reverse) |
| | | | | TCGATGATGCAATCATTCCT |
| | | | | (Sequence Number 184) |
| 93 | NTRK1 | No | NM_001012331 | (Forward) |
| | | | | TCTGGAGCTCCGTGATCTG |
| | | | | (Sequence Number 185) |
| | | | | (Reverse) |
| | | | | AGTGAAATGGAAGGCATCTG |
| | | | | G (Sequence Number 186) |
| 94 | PARP1 | No | NM_001618 | (Forward) |
| | | | | ACTTTGCTGGGATCCTGTCC |
| | | | | (Sequence Number 187) |
| | | | | (Reverse) |
| | | | | CAAACATGTAGCCTGTCACGG |
| | | | | (Sequence Number 188) |
| 95 | PARP2 | No | NM_005484 | (Forward) |
| | | | | TCACAGGTTACATGTTTGGGA |
| | | | | (Sequence Number 189) |
| | | | | (Reverse) |
| | | | | TTCATTACACTGACCTAGAGC |
| | | | | TACC (Sequence Number 190) |
| 96 | PARP3 | No | NM_001003931 | (Forward) |
| | | | | CAAGAACAACTGGGCAGAGC |
| | | | | (Sequence Number 191) |
| | | | | (Reverse) |
| | | | | GGCCTCTGTCCACCTTCAC |
| | | | | (Sequence Number 192) |
| 97 | PDCD1 | No | NM_005018 | (Forward) |
| | | | | ATAGGAGCCAGGCGCA |
| | | | | (Sequence Number 193) |
| | | | | (Reverse) |
| | | | | CCAGCTCCCCATAGTCCA |
| | | | | (Sequence Number 194) |
| 98 | PGF | No | NM_002632 | (Forward) |
| | | | | GAGCTGACGTTCTCTCAGCA |
| | | | | (Sequence Number 195) |
| | | | | (Reverse) |
| | | | | TACCTCCGGGGAACAGCAT |
| | | | | (Sequence Number 196) |
| 99 | PIK3CD | No | NM_005026 | (Forward) |
| | | | | CAGGCATGAGTACCTGTATGG |
| | | | | C(Sequence Number 197) |
| | | | | (Reverse) |
| | | | | CATGGTCAGGTGAGGGGT |
| | | | | (Sequence Number 198) |
| 100 | PRLR | No | NM_000949 | (Forward) |
| | | | | TGGTTCACGCTCCTGTATGA |
| | | | | (Sequence Number 199) |
| | | | | (Reverse) |
| | | | | CTCTGTTTGCTGCCCAGC |
| | | | | (Sequence Number 200) |
| 101 | PSMB10 | No | NM_002801 | (Forward) |
| | | | | ACGGTCACTCGCATCCTG |
| | | | | (Sequence Number 201) |
| | | | | (Reverse) |
| | | | | GGTCCAGTCAGGTCTACGCC |
| | | | | (Sequence Number 202) |
| 102 | PSMB1 | No | NM_002793 | (Forward) |
| | | | | CAATTCATACGCGGGATAGC |
| | | | | (Sequence Number 203) |
| | | | | (Reverse) |
| | | | | CCATGAAAACCGCTGCAT |
| | | | | (Sequence Number 204) |
| 103 | PSMB2 | No | NM_002794 | (Forward) |
| | | | | TCACACGCCGAAACCTG |
| | | | | (Sequence Number 205) |
| | | | | (Reverse) |
| | | | | GGCCCTTCATGCTCATCATAG |
| | | | | (Sequence Number 206) |
| 104 | PSMB8 | No | NM_004159 | (Forward) |
| | | | | GCAGCAGTGGATTCTCGG |
| | | | | (Sequence Number 207) |
| | | | | (Reverse) |
| | | | | GCAGGTAAGGGTTAATCTCAA |
| | | | | TCA (Sequence Number 208) |
| 105 | PSMB9 | No | NM_002800 | (Forward) |
| | | | | CATGGTAGCTGGCTGGGA |
| | | | | (Sequence Number 209) |
| | | | | (Reverse) |
| | | | | AAGGCTGTCGAGTCAGCATT |
| | | | | (Sequence Number 210) |
| 106 | PSMD1 | No | NM_002807 | (Forward) |
| | | | | GGGACCTCTTCAATGTCAATG |
| | | | | AT (Sequence Number 211) |
| | | | | (Reverse) |
| | | | | TCCACACATTGTTTGGTGTAG |
| | | | | TG (Sequence Number 212) |
| 107 | PSMD2 | No | NM_002808 | (Forward) |
| | | | | GGTGCCTGATGACATCTACAA |
| | | | | A (Sequence Number 213) |
| | | | | (Reverse) |
| | | | | GCAGAGTCCACCTGAGAGC |
| | | | | (Sequence Number 214) |
| 108 | PTK6 | No | NM_005975 | (Forward) |
| | | | | GATCAGGGTCAGCGAGAAGC |
| | | | | (Sequence Number 215) |
| | | | | (Reverse) |
| | | | | CCGCCAGATCTTGTAGTGCC |
| | | | | (Sequence Number 216) |
| 109 | RARA | No | NM_000964 | (Forward) |
| | | | | TTAGCCTGCCCTCTTTGGAC |
| | | | | (Sequence Number 217) |
| | | | | (Reverse) |
| | | | | TCAGAAGGGAGGCAGACAGT |
| | | | | (Sequence Number 218) |
| 110 | RARB | No | NM_000965 | (Forward) |
| | | | | AGCAAGCCTCACATGTTTCC |
| | | | | (Sequence Number 219) |
| | | | | (Reverse) |
| | | | | CAAGGTAATTACACGCTCTGC |
| | | | | (Sequence Number 220) |
| 111 | RARG | No | NM_000966 | (Forward) |
| | | | | AGCTCATCACCAAGGTCAGC |
| | | | | (Sequence Number 221) |
| | | | | (Reverse) |
| | | | | TCGTGGTATACTTGCCCAGC |
| | | | | (Sequence Number 222) |
| 112 | RPL3 | No | NM_000967 | (Forward) |
| | | | | TACTGCCAAGTCATCCGTGTC |
| | | | | (Sequence Number 223) |
| | | | | (Reverse) |
| | | | | TCTCCATCAGGTGGGCCTTC |
| | | | | (Sequence Number 224) |
| 113 | SH2B3 | No | NM_005475 | (Forward) |
| | | | | CCTGATGCTCATGGAGTGTTC |
| | | | | C (Sequence Number 225) |
| | | | | (Reverse) |
| | | | | GGAAAGTGGAGGTGCTGCAC |
| | | | | AC (Sequence Number 226) |
| 114 | SIK1 | No | NM_173354 | (Forward) |
| | | | | TTCTGCTCCCTGTCAGCTTCC |
| | | | | (Sequence Number 227) |
| | | | | (Reverse) |
| | | | | TCCCAGAAACCCTTTGGTCCG |
| | | | | (Sequence Number 228) |
| 115 | SLC2A2 | No | NM_000340 | (Forward) |
| | | | | ATGTCAGTGGGACTTGTGCTG |
| | | | | C (Sequence Number 229) |
| | | | | (Reverse) |
| | | | | AACTCAGCCACCATGAACCA |
| | | | | GG (Sequence Number 230) |
| 116 | SMO | No | NM_005631 | (Forward) |
| | | | | GAAGTGCCCTTGGTTCGGA |
| | | | | (Sequence Number 231) |
| | | | | (Reverse) |
| | | | | GCAGGGTAGCGATTCGAGTT |
| | | | | (Sequence Number 232) |
| 117 | SSTR2 | No | NM_001050 | (Forward) |
| | | | | ACTCAATGGAAGCCACACATG |
| | | | | G (Sequence Number 233) |
| | | | | (Reverse) |
| | | | | TACGGCTCTGTCTGGTTTGAG |
| | | | | G (Sequence Number 234) |
| 118 | SSTR5 | No | NM_001053 | (Forward) |
| | | | | CGTGGTCATCCTCTCCTACG |
| | | | | (Sequence Number 235) |
| | | | | (Reverse) |
| | | | | ACCTTCTGGAAGCTCTGGC |
| | | | | (Sequence Number 236) |
| 119 | TEK | No | NM_000459 | (Forward) |
| | | | | GTTGGCCTTTCTGATCATATTG |
| | | | | C (Sequence Number 237) |
| | | | | (Reverse) |
| | | | | CTGCACAGCTGGTTCTTCCC |
| | | | | (Sequence Number 238) |
| 120 | TLR8 | No | NM_016610 | (Forward) |
| | | | | ATGTTCCTTCAGTCGTCAATG |
| | | | | C (Sequence Number 239) |
| | | | | (Reverse) |
| | | | | TTGCTGCACTCTGCAATAACT |
| | | | | (Sequence Number 240) |
| 12 | TNFSF8 | No | NM_001244 | (Forward) |
| | | | | ATCTGTGCCACATCAGCCACC |
| | | | | A (Sequence Number 241) |
| | | | | (Reverse) |
| | | | | AAGGTGGTGTCCTTCTCAGCC |
| | | | | A (Sequence Number 242) |
| 122 | TNFSF11 | No | NM_003701 | (Forward) |
| | | | | CAGCACATCAGAGCAGAGAA |
| | | | | AG (Sequence Number 243) |
| | | | | (Reverse) |
| | | | | AGCAAAAGGCTGAGCTTCAA |
| | | | | (Sequence Number 244) |
| 123 | TOP1MT | No | NM_052963 | (Forward) |
| | | | | CAGGGAGGAGAAGCAGAAGC |
| | | | | (Sequence Number 245) |
| | | | | (Reverse) |
| | | | | AGGCGGCTCAATCTTGAAGT |
| | | | | (Sequence Number 246) |
| 124 | TOP2B | No | NM_001330700 | (Forward) |
| | | | | ATGTCAGACGAATGCTAGATG |
| | | | | G (Sequence Number 247) |
| | | | | (Reverse) |
| | | | | CCAAGTTCTTGAATCGTGCC |
| | | | | (Sequence Number 248) |
| 125 | TUBA1A | No | NM_006009 | (Forward) |
| | | | | GCGTTTTGGAAAGATACCCA |
| | | | | (Sequence Number 249) |
| | | | | (Reverse) |
| | | | | TTGCCAATCTGGACACCA |
| | | | | (Sequence Number 250) |
| 126 | TUBA4A | No | NM_006000 | (Forward) |
| | | | | AGCTCTATTGCTTGGAACATG |
| | | | | GG (Sequence Number 251) |
| | | | | (Reverse) |
| | | | | CCCCTCCACCAATGGTCTTGT |
| | | | | (Sequence Number 252) |
| 127 | TUBB1 | No | NM_030773 | (Forward) |
| | | | | GCGTGAAATTGTCCATATTCA |
| | | | | GA (Sequence Number 253) |
| | | | | (Reverse) |
| | | | | AATCATCTCCCAGAACTTGGC |
| | | | | T (Sequence Number 254) |
| 128 | TUBB3 | No | NM_006086 | (Forward) |
| | | | | GCCTGACAATTTCATCTTTGG |
| | | | | TCA (Sequence Number 255) |
| | | | | (Reverse) |
| | | | | CAGGCAGTCGCAGTTTTCAC |
| | | | | (Sequence Number 256) |
| 129 | TUBB | No | NM_001293212 | (Forward) |
| | | | | CAGCTGGACCGCATCTCT |
| | | | | (Sequence Number 257) |
| | | | | (Reverse) |
| | | | | AGATCCACCAGGATGGCA |
| | | | | (Sequence Number 258) |
| 130 | TUBD1 | No | NM_016261 | (Forward) |
| | | | | ATATGATTGTTGGGAAGGCAT |
| | | | | G (Sequence Number 259) |
| | | | | (Reverse) |
| | | | | AAGTCCTCTTCTTCGATTCCA |
| | | | | A (Sequence Number 260) |
| 131 | TUBE1 | No | NM_016262 | (Forward) |
| | | | | TTCTGGGACCTGGCACTAAG |
| | | | | (Sequence Number 261) |
| | | | | (Reverse) |
| | | | | ATCACCAACCACTCTGGTGTC |
| | | | | (Sequence Number 262) |
| 132 | TUBG1 | No | NM_001070 | (Forward) |
| | | | | GGAGAAAAGATCCATGAGGA |
| | | | | CA (Sequence Number 263) |
| | | | | (Reverse) |
| | | | | CCCAGCAATGGAGTGACA |
| | | | | (Sequence Number 264) |
| 133 | VEGFA | No | NM_001025366 | (Forward) |
| | | | | CTTGCCTTGCTGCTCTACCT |
| | | | | (Sequence Number 265) |
| | | | | (Reverse) |
| | | | | TCCATGAACTTCACCACTTCG |
| | | | | T (Sequence Number 266) |
| 134 | VEGFB | No | NM_001243733 | (Forward) |
| | | | | CTGACGATGGCCTGGAGTG |
| | | | | (Sequence Number 267) |
| | | | | (Reverse) |
| | | | | CGGGTACCGGATCATGAGGA |
| | | | | (Sequence Number 268) |

Using the above primers, ddPCR reaction mixtures were prepared with the composition listed in [Table 3]. A volume of 12 μl of the ddPCR reaction mixture was dispensed into the wells of a nano plate, and ddPCR was performed using the QIAcuity One ddPCR system under the conditions specified in [Table 4]. The quantity of the genes was quantified using the QuantaSoft 5 program.

TABLE 3

| Singleplex | 1X (μl) |
|---|---|
| 3X EG PCR Master mix (FAM) | 4 |
| 10X Primer Mix | 0.96 |
| RNase-free water | 6.04 |
| Template | 1 |

TABLE 4

| PCR condition | | | | |
|---|---|---|---|---|
| Step | Time | Temp | | Imaging condition |
| PCR initial heat | 2 min | 95° C. | | Green channel, |
| activation | | | | exposure in 200 ms |
| Denaturation | 15 sec | 95° C. | 40 cycle | |
| Annealing | 15 sec | 55-62° C. | | |
| Extension | 15 sec | 72° C. | | |
| Cooling down | 5 min | 1° C. | | |

### <Comparative Example 1> Quantitative Real-Time PCR Analysis

Using the cDNA obtained in Example 1 as a template, quantitative real-time PCR (qPCR) analysis was performed with the primers listed in [Table 1] and [Table 2].

Specifically, the qPCR reaction mixture was prepared with the composition listed in [Table 5]. A volume of 10 μl of the qPCR reaction mixture was dispensed into a 384-well plate, and qPCR was performed using the ABI 7900 HT system under the conditions specified in [Table 6]. The quantity of the genes was quantified using the SDS 2.4 software program.

TABLE 5

| reaction solution | 1X (pl) |
|---|---|
| 2X SYBR | 5 |
| 0.25 μM Primer Mix | 1.2 |
| RNase-free water | 2.8 |
| Template | 1 |

TABLE 6
PCR condition

| Stage | Step | Time | | Temp |
|---|---|---|---|---|
| Hold stage | 1 | 2 min | | 50° C. |
| | 2 (Initial heating) | 10 min | | 95° C. |
| PCR stage | 1 (Denaturation) | 15 sec | 40 cycles | 95° C. |
| | 2 (Annealing) | 1 min | | 60° C. |

| | | | |
|---|---|---|---|
| Melt curve stage | 1 | 15 sec | 95° C. |
| | 2 | 1 min | 60° C. |
| | 3 (Dissociation) | 15 sec | 95° C. |

### <Experimental Example 1> Target Gene Expression Profile Through Digital PCR Analysis

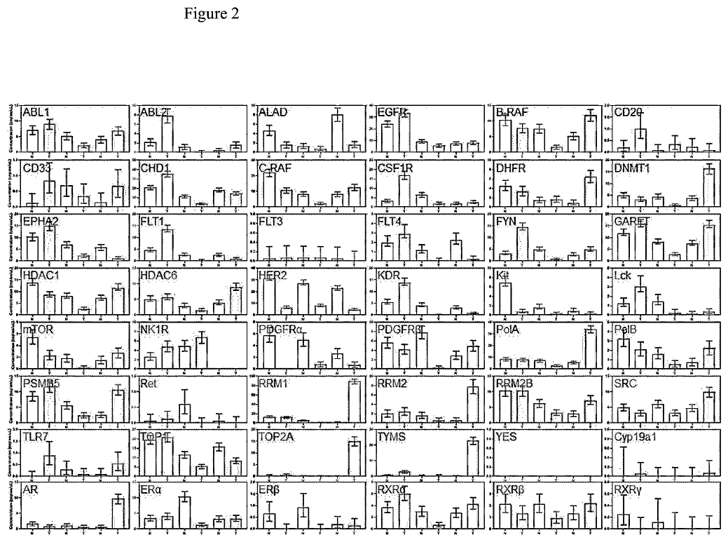

Using normal and cancer tissue samples obtained from three renal cancer patients, ddPCR was performed according to the method described in Example 2 to complete the expression profile of 48 target genes (see FIG. 2).

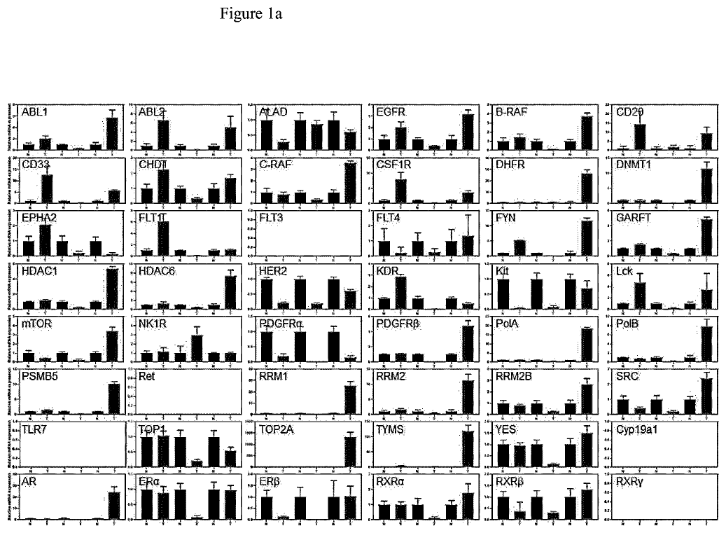

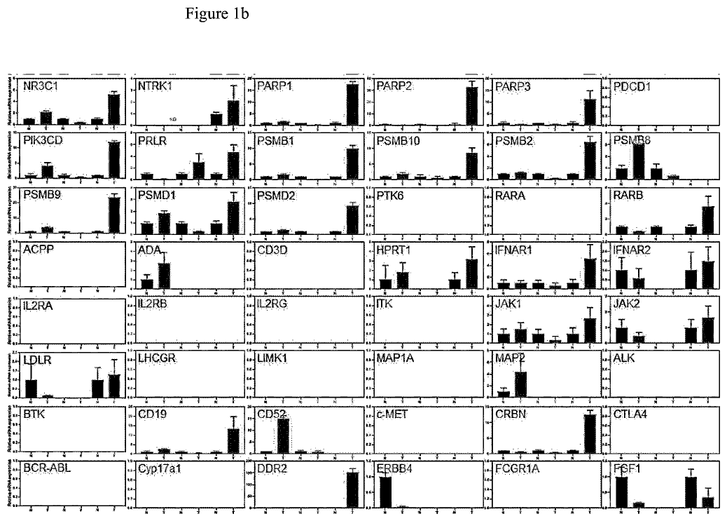

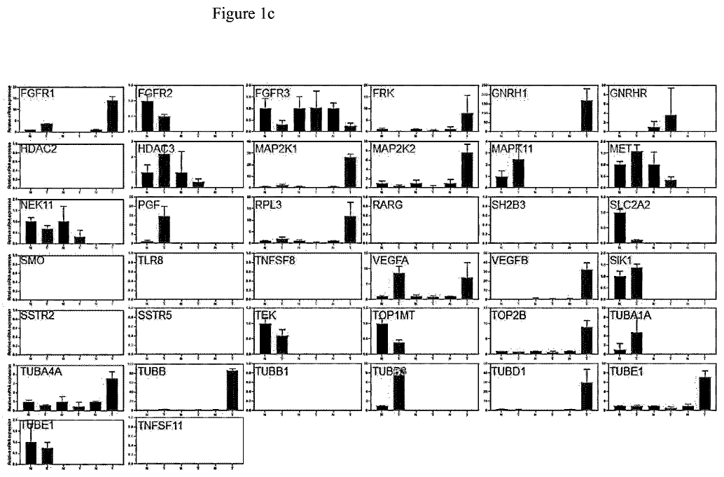

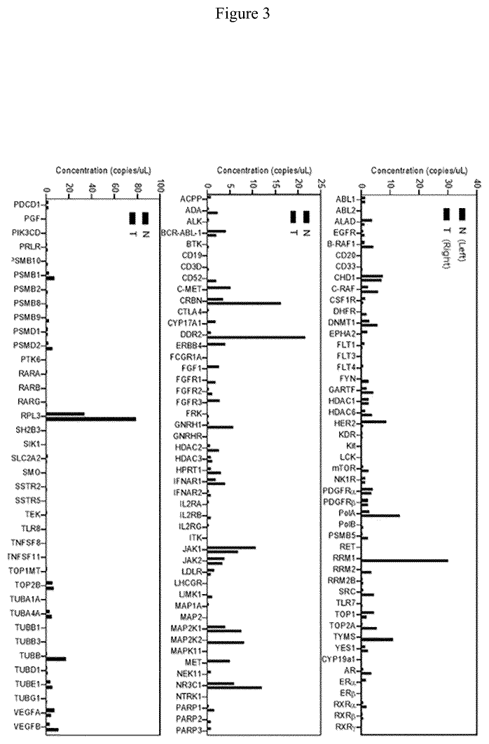

Additionally, using one pair of normal and cancer tissue samples from the three renal cancer patients (specifically, sample 3 from FIG. 1a to FIG. 1c and FIG. 2), ddPCR was performed according to the method described in Example 2 to complete the expression profile of 134 target genes (see FIG. 3).

Furthermore, using the normal and cancer tissue samples obtained from the three renal cancer patients, qPCR was performed according to the method described in Comparative Example 1 to complete the expression profile of 134 target genes (see FIG. 1a to FIG. 1c).

As a result, it was confirmed that there are differences in the expression patterns of the 134 target genes between the normal and cancer tissues of the renal cancer patients, as shown in FIGS. 1a to 1c, FIG. 2, and FIG. 3. In particular, when ddPCR and qPCR were performed on the 48 target genes, ddPCR revealed more distinct expression patterns for the target genes compared to qPCR, with notably clearer expression observed for genes such as PSMB5, PDGFRβ, and POlB (see FIGS. 1a and 2).

Additionally, it was confirmed that ddPCR can be used to obtain expression profiles of 134 target genes in normal and cancer tissues of renal cancer patients. Notably, 23 target genes, such as ADA, B-RAF, CD52, DNMT1, HPRT1, PolA, PSMB1, PSMB5, RRM1, RRM2, SRC, TOP2A, TYMS, AR, PARP1, PARP2, CRBN, DDR2, FGFR1, GNRH1, MAP2K2, VEGFB, and TUBB, exhibited significantly higher expression levels, showing over a 5-fold difference in cancer tissues compared to normal tissues. Particularly, for the target genes ADA and CD52, while it was difficult to confirm the expression patterns using qPCR, clear expression patterns were obtained through ddPCR (see FIGS. 1a to 1c and FIG. 3).

The results indicate that even within the same type of renal cancer, the expression of cancer therapeutic target genes varies among patients. This suggests that by understanding the individual expression patterns for each patient, effective treatment strategies can be developed.

Therefore, the present invention analyzes the expression patterns of 134 cancer therapeutic target genes between the normal and cancer tissues of cancer patients and confirms that selective administration of existing or future-developed cancer therapies based on these expression patterns can lead to personalized treatment tailored to individual patients.

### INDUSTRIAL APPLICABILITY

In the present invention, expression changes of 134 cancer therapeutic target genes between normal and cancer tissues in cancer patients are quantified using digital PCR. By analyzing this expression accurately and efficiently, it has been confirmed that there are differences in the expression patterns of target genes among cancer patients. Therefore, a kit comprising primers or probes specific to the 134 cancer therapeutic target genes can be utilized to selectively administer existing or future-developed cancer therapeutic agents to cancer patients, enabling personalized treatment tailored to each patient.

1. A kit for screening personalized therapeutic agents for cancer patients, comprising primers or probes that specifically hybridize to the target genes ADA and CD52; and primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ.

2. The kit for screening personalized therapeutic agents for cancer patients according to claim 1, wherein the targets essentially include ADA and CD52, and comprise at least 10 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ.

3. The kit for screening personalized therapeutic agents for cancer patients according to claim 2, wherein the targets essentially include ADA and CD52, and comprise at least 20 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ.

4. The kit for screening personalized therapeutic agents for cancer patients according to claim 3, wherein the targets essentially include ADA and CD52, and comprise at least 25 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ.

5. The kit for screening personalized therapeutic agents for cancer patients according to claim 4, wherein the targets essentially include ADA and CD52, and comprise at least 35 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ.

6. The kit for screening personalized therapeutic agents for cancer patients according to claim 5, wherein the targets essentially include ADA and CD52, and comprise at least 45 targets selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ.

7. The kit for screening personalized therapeutic agents for cancer patients according to claim 1, wherein the primers that specifically hybridize to each of the target genes are selected from the group consisting of primers designated by sequence numbers 1 to 96, 99, 100, 111, and 112.

8. The kit for screening personalized therapeutic agents for cancer patients according to claim 1, further comprising primers or probes that specifically hybridize to at least 10 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

9. The kit for screening personalized therapeutic agents for cancer patients according to claim 8, further comprising primers or probes that specifically hybridize to at least 20 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

10. The kit for screening personalized therapeutic agents for cancer patients according to claim 9, further comprising primers or probes that specifically hybridize to at least 40 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

11. The kit for screening personalized therapeutic agents for cancer patients according to claim 10, further comprising primers or probes that specifically hybridize to at least 60 target genes selected from the group consisting of ACPP, ADA, ALK, BCR-ABL-1, BTK, CD19, CD30, CD52, C-MET, CRBN, CTLA4, CYP17A1, DDR2, ERBB4, FCGR1A, FGF1, FGFR1, FGFR2, FGFR3, FRK, GNRH1, GNRHR, HDAC2, HDAC3, HPRT1, IFNAR1, IFNAR2, IL2RA, IL2RB, IL2RG, ITK, JAK1, JAK2, LDLR, LHCGR, LIMK1, MAP1A, MAP2, MAP2K1, MAP2K2, MAPK11, MET, NEK11, NR3C1, NTRK1, PARP1, PARP2, PARP3, PDCD1, PGF, PIK3CD, PRLR, PSMB10, PSMB1, PSMB2, PSMB8, PSMB9, PSMD1, PSMD2, PTK6, RARA, RARB, RARG, RPL3, SH2B3, SIK1, SLC2A2, SMO, SSTR2, SSTR5, TEK, TLR8, TNFSF8, TNFSF11, TOP1MT, TOP2B, TUBA1A, TUBA4A, TUBB1, TUBB3, TUBB, TUBD1, TUBE1, TUBG1, VEGFA, and VEGFB.

12. The kit for screening personalized therapeutic agents for cancer patients according to claim 8, wherein the primers that specifically hybridize to each of the target genes are selected from the group consisting of primers designated by sequence numbers 97, 98, 101 to 110, and 113 to 268.

13. The kit for screening personalized therapeutic agents for cancer patients according to claim 1, characterized in that the kit is a digital PCR kit.

14. The kit for screening personalized therapeutic agents for cancer patients according to claim 1, characterized in that the kit is a droplet digital PCR kit.

15. The kit for screening personalized therapeutic agents for cancer patients according to claim 1, characterized in that the cancer is selected from the group consisting of renal cancer, gastric cancer, lung cancer, breast cancer, ovarian cancer, liver cancer, bronchial cancer, nasopharyngeal cancer, laryngeal cancer, pancreatic cancer, bladder cancer, colorectal cancer, colon cancer, cervical cancer, brain cancer, prostate cancer, bone cancer, head and neck cancer, skin cancer, thyroid cancer, parathyroid cancer, ureter cancer, and hematological cancer.

16. A method for screening personalized therapeutic agents for cancer patients, comprising the steps of:
1) measuring the expression of the target genes in a biological sample isolated from a cancer patient, wherein the target genes comprise ADA and CD52 essentially; and at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, Lck, mTOR, NK1R, PDGFRα, PDGFRβ, PolA, PolB, PSMB5, Ret, RRM1, RRM2, RRM2B, SRC, TLR7, TOP1, TOP2A, TYMS, YES, CYP19A1, AR, ERα, ERβ, RXRα, RXRβ, and RXRγ; and
2) screening for drugs that act on the target genes measured to be highly expressed in step 1).

17. The method for screening personalized therapeutic agents for cancer patients according to claim 16, wherein measuring the expression of the genes is performed using a digital PCR method.

18. The method for screening personalized therapeutic agents for cancer patients according to claim 17, wherein measuring the expression of the genes is performed using a droplet digital PCR method.

19. A method for screening personalized therapeutic agents for cancer patients, comprising the following steps:
1) isolating and proliferating cancer cells and normal cells from cancer tissue and normal tissue obtained from a cancer patient;
2) treating the cancer cells from step 1) with a candidate anticancer agent;
3) isolating total RNA from the cancer cells of step 1) and normal cells of step 1), and from the cancer cells treated with the candidate anticancer agent of step 2), and synthesizing cDNA using the total RNA as a template;
4) using the synthesized cDNA of step 3) as a template, generating multiple droplets from a reaction solution containing primers or probes that specifically hybridize to the target genes ADA and CD52, as well as primers or probes that specifically hybridize to at least five target genes selected from the group consisting of ABL1, ABL2, ALAD, EGFR, B-RAF, CD20, CD33, CHD1, C-RAF, CSF1R, DHFR, DNMT1, EPHA2, FLT1, FLT3, FLT4, FYN, GARFT, HDAC1, HDAC6, HER2, KDR, Kit, LCK, mTOR, NK1R, PDGFRα, PDGFRβ, PolA, PolB, PSMB5, RET, RRM1, RRM2, RRM2B, SRC, TLR7, TOP1, TOP2A, TYMS, YES, CYP19A1, AR, ERα, ERβ, RXRα, RXRβ, and RXRγ;
5) amplifying the target nucleic acid sequences in the droplets generated in step 4); and
6) counting the number of droplets containing the amplified target nucleic acids from step 5) to determine the amount of amplified target nucleic acid, followed by comparing the amount of amplified target nucleic acid in normal cells, cancer cells, and cancer cells treated with the candidate anticancer agent.

20. The method for screening personalized therapeutic agents for cancer patients according to claim 19, wherein the primers or probes are designed for use in digital PCR.

21. The method for screening personalized therapeutic agents for cancer patients according to claim 20, wherein the primers or probes are designed for use in droplet digital PCR.

22. A method for screening personalized therapeutic agents for cancer patients according to claim 19, wherein the amount of amplified target nucleic acid of step 6) is increased in cancer cells compared to normal cells, and decreased in cancer cells treated with a candidate therapeutic agent compared to untreated cancer cells, thereby selecting the candidate agent.
